(19) United States
(12) Patent Application Publication
(10) Pub. No.: US 2016/0257648 A1
(43) Pub. Date: Sep. 8, 2016

(54) OXINDOLE INHIBITORS OF TYROSINE KINASE

(71) Applicant: Auspex Pharmaceuticals, Inc., La Jolla, CA (US)

(72) Inventors: Tadimeti Rao, San Diego, CA (US); Chengzhi Zhang, San Diego, CA (US)

(21) Appl. No.: 14/936,213

(22) Filed: Nov. 9, 2015

Related U.S. Application Data
(63) Continuation of application No. 14/245,562, filed on Apr. 4, 2014, now abandoned.

(51) Int. Cl. C07D 209/34; C07B 59/00

(52) U.S. Cl. CPC C07D 209/34; C07B 59/002; C07B 2200/05

(57) ABSTRACT

The present invention relates to new oxindole inhibitors of tyrosine kinase, pharmaceutical compositions thereof, and methods of use thereof.

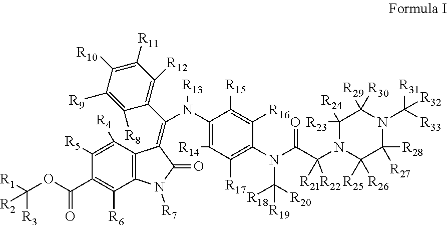

Disclosed herein are new oxindole compounds and compositions and their application as pharmaceuticals for the treatment of disorders. Methods of inhibition of tyrosine kinase activity in a subject are also provided for the treatment of disorders such as non-small cell lung cancer, cancer of the peritoneal cavity, disorders related to the female reproductive system, idiopathic pulmonary fibrosis, colorectal cancer, and prostate cancer.

Nintedanib (Vargatef, BIBF-1120, CAS #656247-17-5), (3Z)-2,3-dihydro-3-[[[4-[methyl[2-(4-methyl-1-piperazinyl)acetyl]amino]phenyl]amino]phenylmethylene]-2-oxo-1H-indole-6-carboxylic acid methyl ester, is a tyrosine kinase inhibitor. Nintedanib is currently under investigation for the treatment of non-small cell lung cancer. Roth et al., J. Med. Chem., 2009, 52(14), 4466-4480; WO 2004017948; WO 2006067165; and U.S. Pat. No. 6,762,180. Nintedanib has also shown promise in treating cancer of the peritoneal cavity, disorders related to the female reproductive system, idiopathic pulmonary fibrosis, colorectal cancer, and prostate cancer. Roth et al., J. Med. Chem., 2009, 52(14), 4466-4480; WO 2004017948; WO 2006067165; and U.S. Pat. No. 6,762,180.

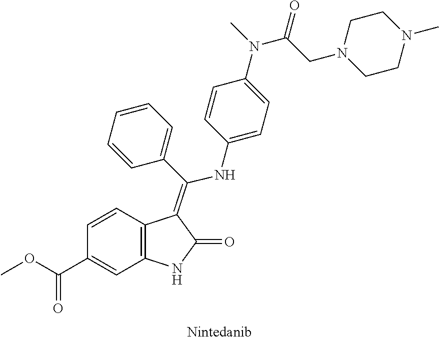

The nintedanib chemical structure contains a number of features that we posit will produce inactive or toxic metabolites, the formation of which can be reduced by the approach described herein. Nintedanib is subject to extensive CYP450-mediated metabolic oxidation. These, as well as other metabolic transformations, occur in part through polymorphically-expressed enzymes, exacerbating interpatient variability. Additionally, some nintedanib metabolites have undesirable side effects. In order to overcome its short half-life, the drug likely must be taken daily, which increases the probability of patient incompliance and discontinuance. Further, abruptly stopping treatment with nintedanib can lead to withdrawal or discontinuation syndrome. Medicines with longer half-lives will likely attenuate these deleterious effects.

### Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C—¹H bond. If a C—¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C—¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Nintedanib is a tyrosine kinase inhibitor. The carbon-hydrogen bonds of Nintedanib contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could effect the pharmacokinetic, pharmacologic and/or toxicologic profiles of such Nintedanib in comparison with the compound having naturally occurring levels of deuterium.

Based on discoveries made in our laboratory, as well as considering the literature, nintedanib is likely metabolized in humans at the N-methyl groups, the N-methylene group, and the piperazine ring. The current approach has the potential to prevent metabolism at these sites. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. All of these transformations can occur through polymorphically-expressed enzymes, exacerbating interpatient variability. Further, some disorders are best treated when the subject is medicated around the clock or for an extended period of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of nintedanib and attenuate interpatient variability.

Novel compounds and pharmaceutical compositions, certain of which have been found to inhibit tyrosine kinase have been discovered, together with methods of synthesizing and using the compounds, including methods for the treatment of tyrosine kinase-mediated disorders in a patient by administering the compounds.

In certain embodiments of the present invention, compounds have structural Formula I:

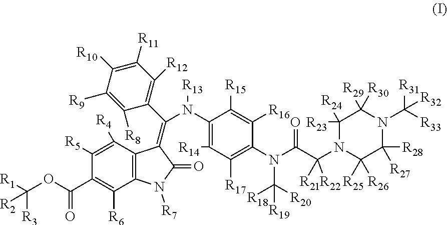

or a salt thereof, wherein:

R₁-R₃₃ are independently selected from the group consisting of hydrogen and deuterium; and

at least one of R₁-R₃₃ is deuterium.

In certain embodiments, if R₃₁-R₃₃ are each deuterium, at least one of R₁-R₃₀ is deuterium.

Certain compounds disclosed herein may possess useful tyrosine kinase inhibiting activity, and may be used in the treatment or prophylaxis of a disorder in which tyrosine kinase plays an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for inhibiting tyrosine kinase. Other embodiments provide methods for treating a tyrosine kinase-mediated disorder in a patient in need of such treatment, comprising administering to said patient a therapeutically effective amount of a compound or composition according to the present invention. Also provided is the use of certain compounds disclosed herein for use in the manufacture of a medicament for the prevention or treatment of a disorder ameliorated by the inhibition of tyrosine kinase.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, at least one of R₁-R₃₃ independently has deuterium enrichment of no less than about 10%.

In certain embodiments, at least one of R₁-R₃₃ independently has deuterium enrichment of no less than about 50%.

In certain embodiments, at least one of R₁-R₃₃ independently has deuterium enrichment of no less than about 90%.

In certain embodiments, at least one of R₁-R₃₃ independently has deuterium enrichment of no less than about 98%.

In certain embodiments, compounds disclosed herein have a structural formula selected from the group consisting of

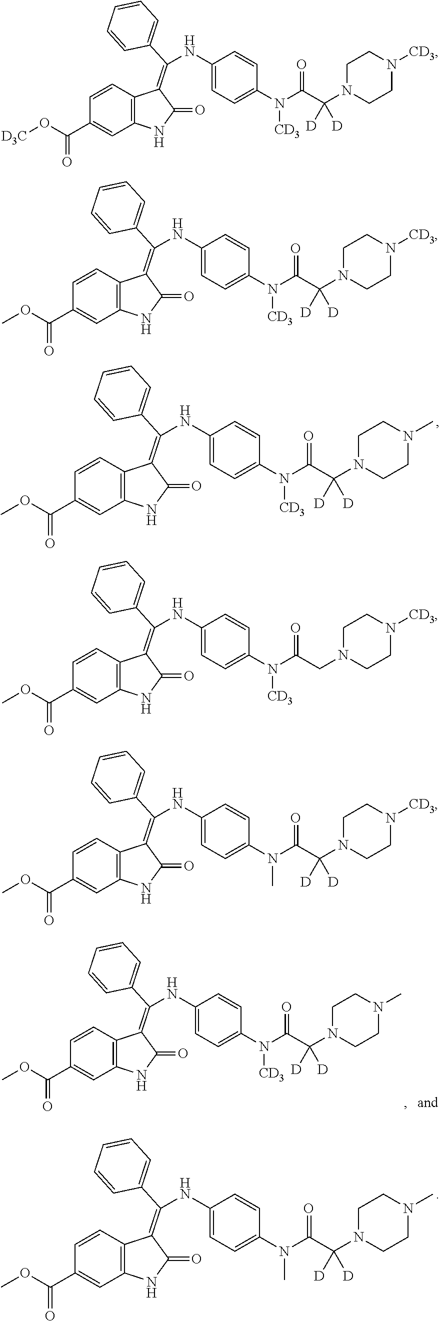

In certain embodiments, each position represented as D has deuterium enrichment of no less than about 10%.

In certain embodiments, each position represented as D has deuterium enrichment of no less than about 50%.

In certain embodiments, each position represented as D has deuterium enrichment of no less than about 90%.

In certain embodiments, each position represented as D has deuterium enrichment of no less than about 98%.

In certain embodiments, compounds disclosed herein have the structural formula:

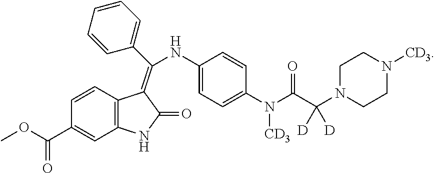 [SMILES: [2H]C([2H])([2H])N1CCN(C([2H])([2H])C(=O)N(c2ccc(N/C(=C3\C(=O)Nc4cc(C(=O)OC)ccc43)c3ccccc3)cc2)C([2H])([2H])[2H])CC1]

In certain embodiments, compounds disclosed herein have the structural formula:

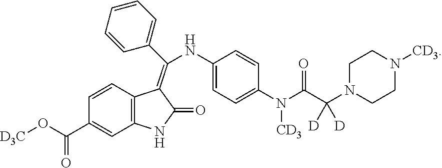 [SMILES: [2H]C([2H])([2H])OC(=O)c1ccc2c(c1)NC(=O)/C2=C(\Nc1ccc(N(C(=O)C([2H])([2H])N2CCN(C([2H])([2H])[2H])CC2)C([2H])([2H])[2H])cc1)c1ccccc1]

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

In certain embodiments, disclosed herein is an extended-release pharmaceutical formulation comprising, in a solid dosage form for oral delivery of between about 100 mg and about 1 g total weight:
between about 2 and about 18% of a compound as disclosed herein;
between about 70% and about 96% of one or more diluents;
between about 1% and about 10% of a water-soluble binder; and
between about 0.5 and about 2% of a surfactant.

In certain embodiments, the diluent or diluents are chosen from mannitol, lactose, and microcrystalline cellulose; the binder is a polyvinylpyrrolidone; and the surfactant is a polysorbate.

In certain embodiments, the extended-release pharmaceutical formulation comprises between about 2.5% and about 11% of a compound as disclosed herein.

In certain embodiments, the extended-release pharmaceutical formulation comprises:
between about 60% and about 70% mannitol or lactose;
between about 15% and about 25% microcrystalline cellulose
about 5% of polyvinylpyrrolidone K29/32; and
between about 1 and about 2% of Tween 80.

In certain embodiments, the extended-release pharmaceutical formulation comprises:
between about 4% and about 9% of a compound as disclosed herein;
between about 60% and about 70% mannitol or lactose;
between about 20% and about 25% microcrystalline cellulose
about 5% of polyvinylpyrrolidone K29/32; and
about 1.4% of Tween 80.

In certain embodiments, disclosed herein is an extended-release pharmaceutical formulation comprising, in a solid dosage form for oral delivery of between about 100 mg and about 1 g total weight:
between about 70 and about 95% of a granulation of a compound as disclosed herein, wherein the active ingredient comprises between about 1 and about 15% of the granulation;
between about 5% and about 15% of one or more diluents;
between about 5% and about 20% of sustained-release polymer; and
between about 0.5 and about 2% of a lubricant.

In certain embodiments, the extended-release pharmaceutical formulation comprises:
between about 5% and about 15% of one or more spray-dried mannitol or spray-dried lactose;
between about 5% and about 20% of sustained-release polymer; and
between about 0.5 and about 2% of a magnesium stearate.

In certain embodiments, the sustained-release polymer is chosen from a polyvinyl acetate-polyvinylpyrrolidone mixture and a poly(ethylene oxide) polymer.

In certain embodiments, the sustained-release polymer is chosen from Kollidon® SR, POLYOX® N60K, and Carbopol®.

In certain embodiments, the sustained-release polymer is Kollidon® SR.

In certain embodiments, the sustained-release polymer is POLYOX® N60K.

In certain embodiments, the sustained-release polymer is Carbopol®.

In certain embodiments, the extended-release pharmaceutical formulation comprises from about 5 mg to about 100 mg of a compound as disclosed herein.

In certain embodiments, the compounds disclosed herein can be formulated as extended-release pharmaceutical formulations as described in U.S. patent application Ser. No. 14/030,322, filed Sep. 18, 2013.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

When ranges of values are disclosed, and the notation “from n₁ . . . to n₂” or “n₁-n₂” is used, where n₁ and n₂ are the numbers, then unless otherwise specified, this notation is intended to include the numbers themselves and the range between them. This range may be integral or continuous between and including the end values.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₃₃ or the symbol “D”, when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease” and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “tyrosine kinase” refers to enzymes which are capable of transferring a phosphate group from ATP to a tyrosine residue in a protein. Phosphorylation of proteins by tyrosine kinases is an important mechanism in signal transduction for regulation of enzyme activity and cellular events such as cell survival or proliferation. Specific tyrosine kinases inhibited by the compounds disclosed herein include vascular endothelial growth factor receptor (VEGFR) tyrosine kinases (including VEGFR-1 (Flt-1), VEGFR-2 (FLK-1/KDR), and VEGFR-3 (FLT4)), PDGFR-alpha, PDGFR-beta, FGFR1, FGFR3; and Lck tyrosine kinase. Of particular interest is VEGFR-2, which is a transmembrane receptor PTK expressed primarily in endothelial cells. Activation of VEGFR-2 by VEGF is a critical step in the signal transduction pathway that initiates tumor angiogenesis. VEGF expression maybe constitutive to tumor cells and can also be upregulated in response to certain stimuli. One such stimulus is hypoxia, where VEGF expression is upregulated in both tumor and associated host tissues. The VEGF ligand activates VEGFR-2 by binding to its extracellular VEGF binding site. This leads to receptor dimerization of VEGFRs and autophosphorylation of tyrosine residues at the intracellular kinase domain of VEGFR-2. The kinase domain operates to transfer a phosphate from ATP to the tyrosine residues, thus providing binding sites for signaling proteins downstream of VEGFR-2 leading ultimately to angiogenesis. Consequently, antagonism of the VEGFR-2 kinase domain would block phosphorylation of tyrosine residues and serve to disrupt initiation of angiogenesis. Specifically, inhibition at the ATP binding site of the VEGFR-2 kinase domain would prevent binding of ATP and prevent phosphorylation of tyrosine residues. Such disruption of the pro-angiogenesis signal transduction pathway associated with VEGFR-2 should therefore inhibit tumor angiogenesis and thereby provide a potent treatment for cancer or other disorders associated with inappropriate angiogenesis.

The term “tyrosine kinase-mediated disorder,” refers to a disorder that is characterized by abnormal tyrosine kinase activity. A tyrosine kinase-mediated disorder may be completely or partially mediated by modulating tyrosine kinase. In particular, a tyrosine kinase-mediated disorder is one in which inhibition of tyrosine kinase results in some effect on the underlying disorder e.g., administration of a tyrosine kinase inhibitor results in some improvement in at least some of the patients being treated.

The term “tyrosine kinase inhibitor,” refers to the ability of a compound disclosed herein to alter the function of tyrosine kinase. An inhibitor may block or reduce the activity of tyrosine kinase by forming a reversible or irreversible covalent bond between the inhibitor and tyrosine kinase or through formation of a noncovalently bound complex. Such inhibition may be manifest only in particular cell types or may be contingent on a particular biological event. The term “inhibit” or “inhibition” also refers to altering the function of tyrosine kinase by decreasing the probability that a complex forms between tyrosine kinase and a natural substrate. In some embodiments, inhibition of tyrosine kinase may be assessed using the methods described in Roth et al., J. Med. Chem., 2009, 52(14), 4466-4480; WO 2004017948; WO 2006067165; and U.S. Pat. No. 6,762,180.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed.; (Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

The compositions include those suitable for oral administration. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

In certain embodiments, surfactants are selected from the group consisting of Tween 80, sodium lauryl sulfate, and docusate sodium.

In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

In certain embodiments, sustained release polymers are selected from the group consisting of POLYOX® (poly (ethylene oxide), POLYOX® N60K grade, Kollidon® SR, HPMC, HPMC (high viscosity), HPC, HPC (high viscosity), and Carbopol®.

In certain embodiments, extended/controlled release coating are selected from a group of ethylcellulose polymers, such as ETHOCEL™ and Surelease® Aqueous Ethylcellulose Dispersions.

In certain embodiments, antioxidants are selected from a group consisting of butylated hydroxyanisole (BHA), butylated hydroxytoluene (BHT), sodium ascorbate, and α-tocopherol.

In certain embodiments, tablet coatings are selected from the group of Opadry® 200, Opadry® II, Opadry® fx, Opadry® amb, Opaglos® 2, Opadry® tm, Opadry®, Opadry® NS, Opalux®, Opatint®, Opaspray®, Nutraficient®.

Preferred unit dosage formulations are those containing an effective dose, as herein below recited, or an appropriate fraction thereof, of the active ingredient.

Compounds may be administered orally at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating a tyrosine kinase-mediated disorder comprising administering to a subject having or suspected to have such a disorder, a therapeutically effective amount of a compound as disclosed herein or a pharmaceutically acceptable salt, solvate, or prodrug thereof.

Tyrosine kinase-mediated disorders, include, but are not limited to, solid tumors, non-small cell lung cancer, cancer of the peritoneal cavity, disorders related to the female reproductive system, idiopathic pulmonary fibrosis, colorectal cancer, prostate cancer, inflammatory bowel disease, colitis ulcerosa, Crohn's disease, rheumatoid arthritis, glomerulonephritis, lung fibrosis, psonasis, psonasrs arthritis, hypersensitivity reactions of the skin, atherosclerosis, restenosis, asthma, multiple sclerosis, type 1 diabetes, acute or chronic graft-versus-host disease, allograft or xenograft rejection, fibrosis and remodeling of lung tissue in chronic obstructive pulmonary disease, fibrosis and remodeling of lung tissue in chronic bronchitis, fibrosis and remodeling of lung tissue in emphysema, lung fibrosis and pulmonary diseases with a fibrotic component, fibrosis and remodeling in asthma, fibrosis in rheumatoid arthritis, virally induced hepatic cirrhosis, radiation-induced fibrosis, post angioplasty restenosis, chronic glomerulonephritis, renal fibrosis in patients receiving cyclosporine and renal fibrosis due to high blood pressure, diseases of the skin with a fibrotic component, excessive scarring, idiopathic pulmonary fibrosis, giant cell interstitial pneumonia, sarcodosis, cystic fibrosis, respiratory distress syndrome, drug-induced lung fibrosis, granulomatosis, silicosis, asbestosis, systemic scleroderma, the virally induced hepatic cirrhosis selected from hepatitis C induced hepatic cirrhosis, scleroderma, sarcodosis, systemic lupus, erythematosus, tumours (e.g. plate epithelial carcinoma, astrocytoma, Kaposis sarcoma, glioblastoma, lung cancer, bladder cancer, carcinoma of the neck, melanoma, ovarian cancer, prostate cancer, breast cancer, small-cell lung cancer, glioma, colorectal carcinoma, urogenital cancer and gastrointestinal carcinoma as well as haematological cancers, such as multiple myeloma), haem angioma, angiofibroma, eye diseases (e.g. diabetic retinopathy), neovascular glaucoma, kidney diseases (e.g. glomerulonephritis), diabetic nephropathy, malignant nephrosclerosis, thrombic microangiopathic syndrome, transplant rejections and glomerulopathy, fibrotic diseases (e.g. cirrhosis of the liver), mesangial cell proliferative diseases, arteriosclerosis and damage to the nerve tissue and also for inhibiting the reocclusion of blood vessels after treatment with a balloon catheter, in vascular prosthetics or after the insertion of mechanical devices for keeping blood vessels open (e.g. stents), and/or any disorder which can lessened, alleviated, or prevented by administering a tyrosine kinase inhibitor.

In certain embodiments, a method of treating a tyrosine kinase-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder, (7) prevention of recurrence, or delay of decline or appearance, of abnormal alimentary or hepatic parameters as the primary clinical benefit, or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described by Li et al. Rapid Communications in Mass Spectrometry 2005, 19, 1943-1950, Hughes et al, Xenobiotica 1992, 22(7), 859-69, Varma et al, Journal of Pharmaceutical and Biomedical Analysis 2004, 36(3), 669-674, Massoud et al, Journal of Chromatography, B: Biomedical Sciences and Applications 1999, 734(1), 163-167, Kim et al, Journal of Pharmaceutical and Biomedical Analysis 2003, 31(2), 341-349, and Lindeke et al, Acta Pharmaceutica Suecica 1981, 18(1), 25-34.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al. (British Journal of Clinical Pharmacology, 2000, 49, 343-351). The inhibition of the MAO_{A} isoform is measured by the method of Weyler et al. (J. Biol Chem. 1985, 260, 13199-13207). The inhibition of the MAO_{B} isoform is measured by the method of Uebelhack et al. (Pharmacopsychiatry, 1998, 31, 187-192).

Examples of polymorphically-expressed cytochrome P450 isoforms in a mammalian subject include, but are not limited to, CYP2C8, CYP2C9, CYP2C19, and CYP2D6.

The metabolic activities of liver microsomes, cytochrome P₄₅₀ isoforms, and monoamine oxidase isoforms are measured by the methods described herein.

Examples of improved disorder-control and/or disorder-eradication endpoints, or improved clinical effects include, but are not limited to, serum vascular endothelial growth factor (VEGF) levels, improved progression-free survival, overall survival rate, tumor shrinkage, tumor response rate, increased median overall survival time, improved overall response rate, improved disease control rate, clinical benefit rate as defined by RECIST criteria, change in forced vital capacity, change in pulmonary function parameters, progression to renal failure, reduced proteinuria, progression-free survival, change in shortness-of-breath, change in oxygen saturation during the six minute walk test, change in distance walked during the six minute walk test, tumor volume, and GFR as calculated using the forty-variable Levey equation.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of tyrosine kinase-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more compounds of structural formula II as disclosed in U.S. Pat. No. 8,383,823, which is hereby incorporated by reference in its entirety:

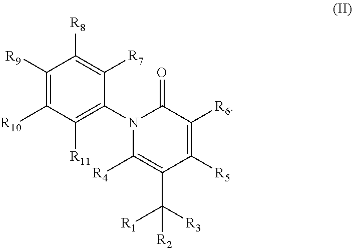

In certain embodiments, the compounds disclosed herein can be combined with a compounds having the structural formula:

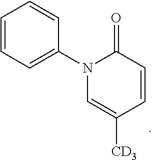 [SMILES: [2H]C([2H])([2H])c1ccc(=O)n(-c2ccccc2)c1]

In certain embodiments, the compounds disclosed herein can be combined with pirfenidone.

In certain embodiments, the compounds disclosed herein can be combined with one or more alkylating agents, anti-metabolite agents, mitotic inhibitors, tyrosine kinase inhibitors, topoisomerase inhibitors, cancer immunotherapy monoclonal antibodies, anti-tumor antibiotic agents, and anti-cancer agents.

In certain embodiments, the compounds disclosed herein can be combined with an alkylating agent selected from the group consisting of chlorambucil, chlormethine, cyclophosphamide, ifosfamide, melphalan, carmustine, fotemustine, lomustine, streptozocin, carboplatin, cisplatin, oxaliplatin, BBR3464, busulfan, dacarbazine, procarbazine, temozolomide, thioTEPA, and uramustine.

In certain embodiments, the compounds disclosed herein can be combined with an anti-metabolite agent selected from the group consisting of aminopterin, methotrexate, pemetrexed, raltitrexed, cladribine, clofarabine, fludarabine, mercaptopurine, pentostatin, tioguanine, cytarabine, fluorouracil, floxuridine, tegafur, carmofur, capecitabine and gemcitabine.

In certain embodiments, the compounds disclosed herein can be combined with a mitotic inhibitor selected from the group consisting of docetaxel, paclitaxel, vinblastine, vincristine, vindesine, and vinorelbine.

In certain embodiments, the compounds disclosed herein can be combined with a tyrosine kinase inhibitor selected from the group consisting of imatinib, BIBW-299, dasatinib, erlotinib, gefitinib, lapatinib, nilotinib, sorafenib, and sunitinib.

In certain embodiments, the compounds disclosed herein can be combined with a topoisomerase inhibitor selected from the group consisting of etoposide, etoposide phosphate, teniposide, camptothecin, topotecan, and irinotecan.

In certain embodiments, the compounds disclosed herein can be combined with a cancer immunotherapy monoclonal antibody selected from the group consisting of rituximab, alemtuzumab, bevacizumab, cetuximab, gemtuzumab, panitumumab, tositumomab, and trastuzumab.

In certain embodiments, the compounds disclosed herein can be combined with an anti-tumor antibiotic agent selected from the group consisting of daunorubicin, doxorubicin, epirubicin, idarubicin, mitoxantrone, valrubicin, actinomycin, bleomycin, mitomycin, plicamycin, and hydroxyurea.

In certain embodiments, the compounds disclosed herein can be combined with an anti-cancer agent selected from the group consisting of amsacrine, asparaginase, altretamine, hydroxycarbamide, lonidamine, pentostatin, miltefosine, masoprocol, estramustine, tretinoin, mitoguazone, topotecan, tiazofurine, irinotecan, alitretinoin, mitotane, pegaspargase, bexarotene, arsenic trioxide, imatinib, denileukin diftitox, bortezomib, celecoxib, and anagrelide.

The compounds disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatgonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating tyrosine kinase-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of tyrosine kinase-mediated disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in Roth et al., J. Med. Chem., 2009, 52(14), 4466-4480; WO 2009071523; WO 2004013099; U.S. Pat. No. 6,762,180, which are hereby incorporated in their entirety, and references cited therein and routine modifications thereof. Compounds as disclosed herein can also be prepared as shown in any of the following schemes and routine modifications thereof.

The following schemes can be used to practice the present invention. Any position shown as hydrogen may optionally be replaced with deuterium.

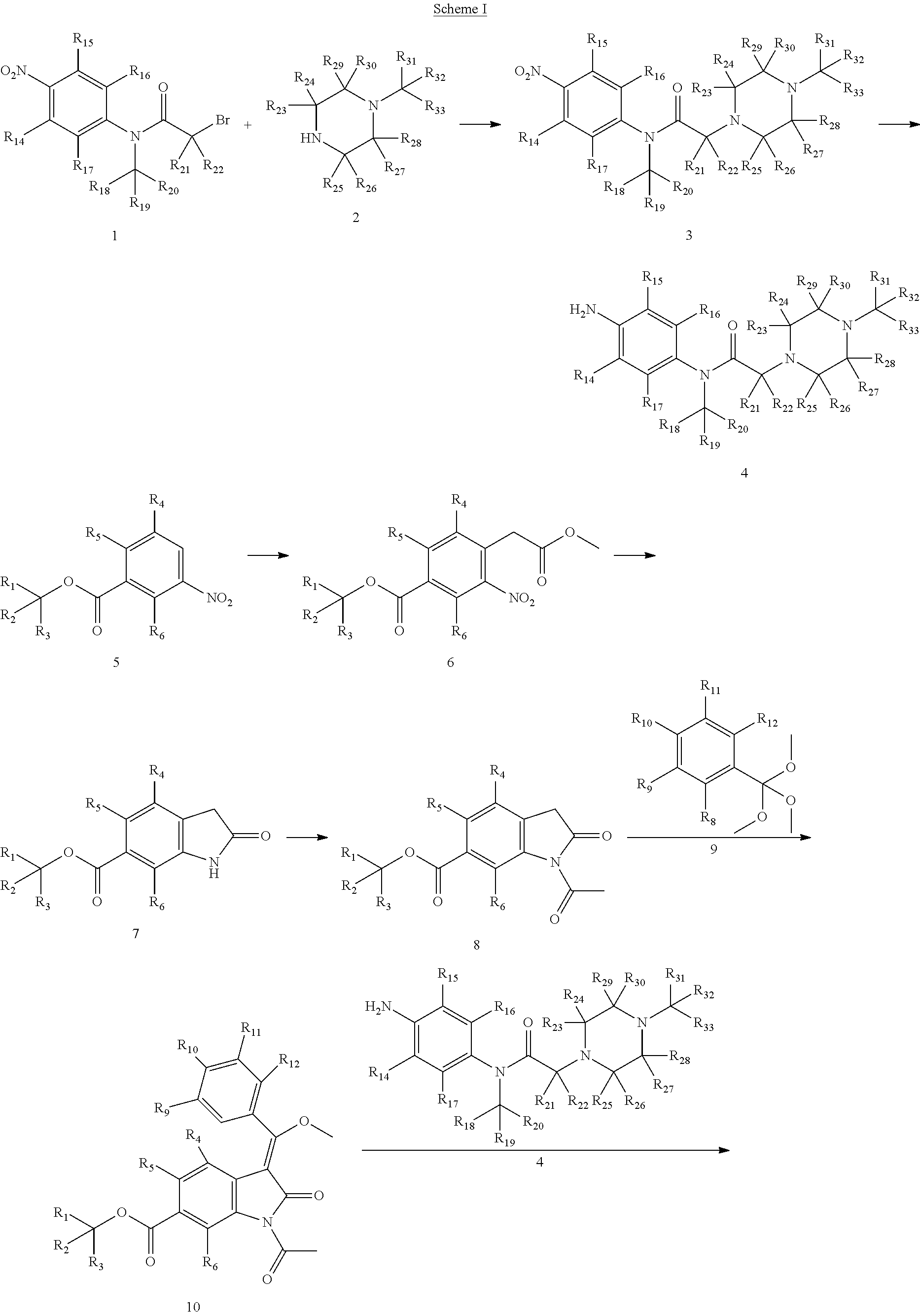

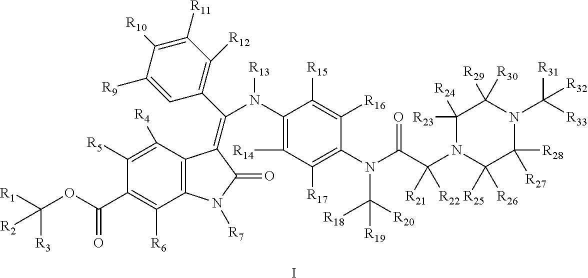

Compound 1 is reacted with compound 2 in the presence of an appropriate base, such as potassium carbonate, in an appropriate solvent, such as acetone, to give compound 3. Compound 3 is treated with an appropriate reducing agent, such as a combination of hydrogen gas and an appropriate catalyst, such as palladium on carbon, in an appropriate solvent, such as methanol, to give compound 4. Compound 5 is reacted with methyl chloroacetate, in the presence of an appropriate base, such as potassium tert-butoxide, in an appropriate solvent, such as dimethylformamide, to give compound 6. Compound 6 is treated with an appropriate reducing agent, such as a combination of hydrogen gas and an appropriate catalyst, such as palladium on carbon, in an appropriate solvent, such as acetic acid, to give compound 7. Compound 7 is reacted with an appropriate acylating agent, such as acetic anhydride, to give compound 8. Compound 8 is reacted with compound 9 in an appropriate solvent, such as acetic anhydride, to give compound 10. Compound 10 is reacted with compound 4 in an appropriate solvent, such as dimethyl formamide, and is the treated with an appropriate base, such as piperidine, to give a compound of formula I.

Deuterium can be incorporated to different positions synthetically, according to the synthetic procedures as shown in Scheme I, by using appropriate deuterated intermediates. For example, to introduce deuterium at one or more positions of R₁₄-R₂₂, compound 1 with the corresponding deuterium substitutions can be used. To introduce deuterium at one or more positions of R₂₃-R₃₃, compound 2 with the corresponding deuterium substitutions can be used. To introduce deuterium at one or more positions of R₁-R₆, compound 5 with the corresponding deuterium substitutions can be used. To introduce deuterium at one or more positions of R₈-R₁₂, compound 9 with the corresponding deuterium substitutions can be used.

Deuterium can be incorporated to various positions having an exchangeable proton, such as the amine N—H and oxindole N—H, via proton-deuterium equilibrium exchange. For example, to introduce deuterium at R₇ or R₁₃, these protons may be replaced with deuterium selectively or non-selectively through a proton-deuterium exchange method known in the art.

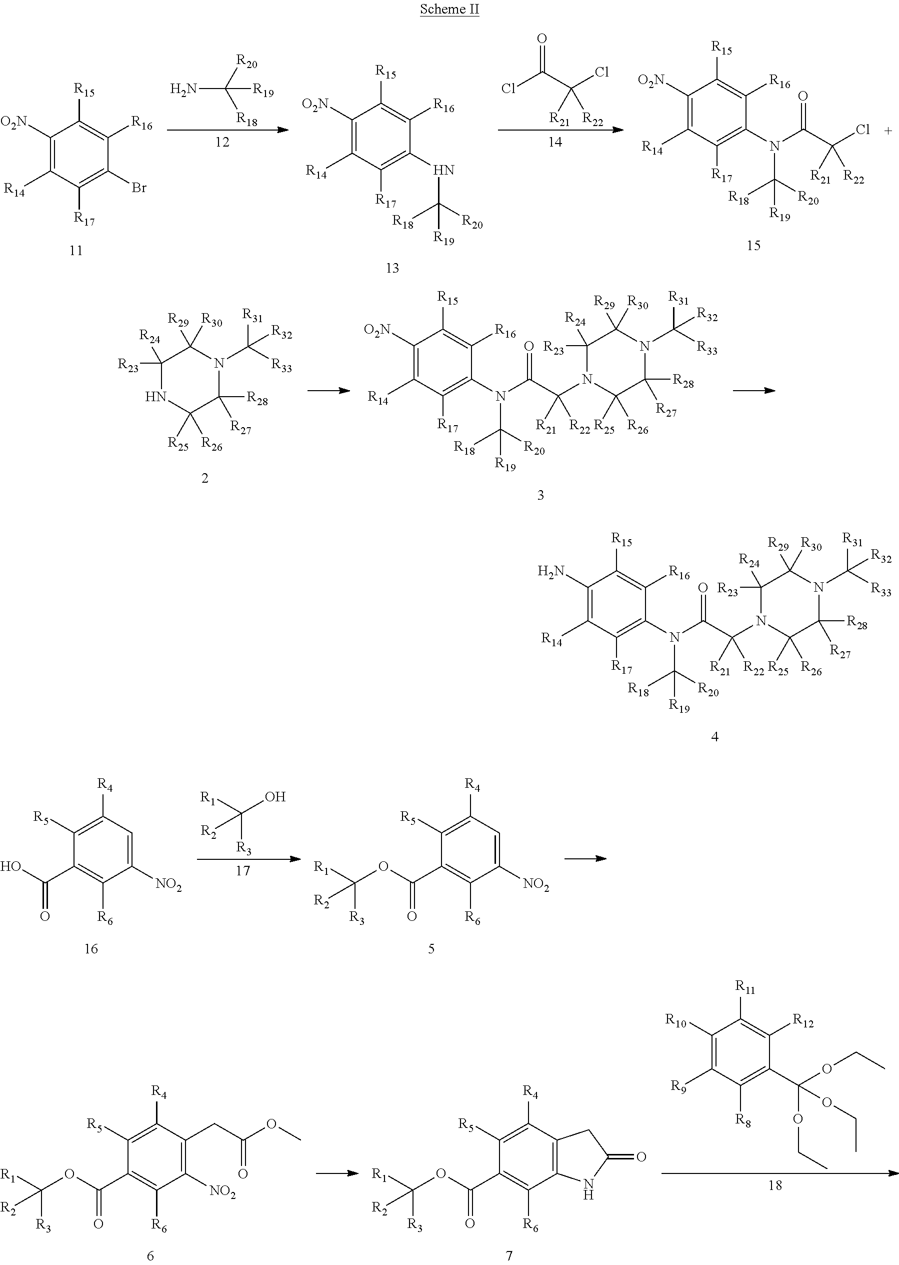

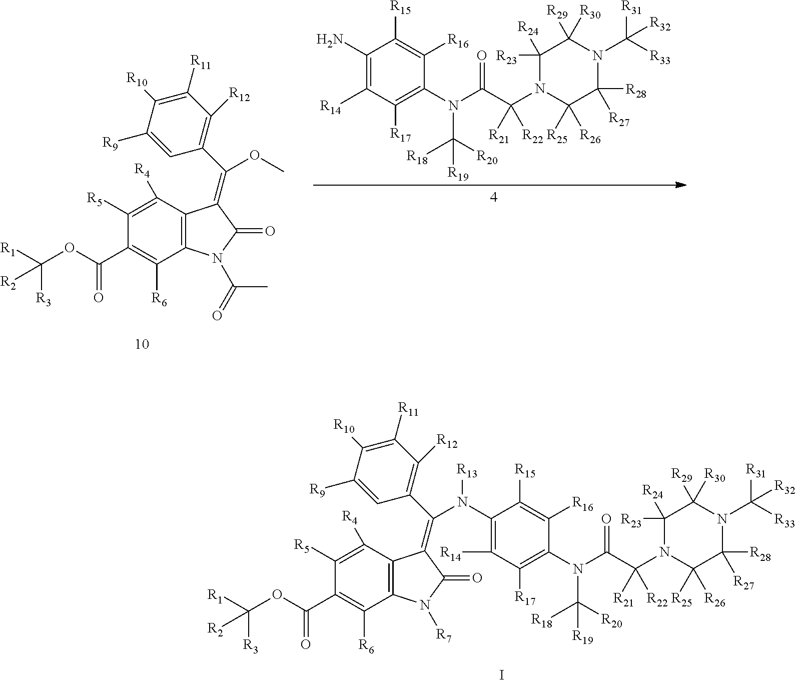

Compound 11 is reacted with compound 12 in an appropriate solvent, such as water, to give compound 13. Compound 13 is reacted with compound 14 in the presence of an appropriate base, such as lithium carbonate, in an appropriate solvent, such as 1,4-dioxane, to give compound 15. Compound 15 is reacted with compound 2 in the presence of an appropriate base, such as potassium carbonate, in an appropriate solvent, such as acetone, to give compound 3. Compound 3 is treated with an appropriate reducing agent, such as a combination of hydrogen gas and an appropriate catalyst, such as palladium on carbon, in an appropriate solvent, such as methanol, to give compound 4. Compound 16 is reacted with compound 17 in the presence of an appropriate acyl activating agent, such as thionyl chloride, to give compound 5. Compound 5 is reacted with methyl chloroacetate, in the presence of an appropriate base, such as potassium tert-butoxide, in an appropriate solvent, such as dimethylformamide, to give compound 6. Compound 6 is treated with an appropriate reducing agent, such as a combination of hydrogen gas and an appropriate catalyst, such as palladium on carbon, in an appropriate solvent, such as acetic acid, to give compound 7. Compound 7 is reacted with compound 18 in an appropriate solvent, such as a combination of acetic anhydride and toluene, to give compound 10. Compound 10 is reacted with compound 4 in an appropriate solvent, such as dimethyl formamide, and is the treated with an appropriate base, such as piperidine, to give a compound of formula I.

Deuterium can be incorporated to different positions synthetically, according to the synthetic procedures as shown in Scheme II, by using appropriate deuterated intermediates. For example, to introduce deuterium at one or more positions of R₁₄-R₁₇, compound 11 with the corresponding deuterium substitutions can be used. To introduce deuterium at one or more positions of R₁₈-R₂₀, compound 12 with the corresponding deuterium substitutions can be used. To introduce deuterium at one or more positions of R₂₁-R₂₂, compound 14 with the corresponding deuterium substitutions can be used. To introduce deuterium at one or more positions of R₂₃-R₃₃, compound 2 with the corresponding deuterium substitutions can be used. To introduce deuterium at one or more positions of R₄-R₆, compound 16 with the corresponding deuterium substitutions can be used. To introduce deuterium at one or more positions of R₁-R₃, compound 17 with the corresponding deuterium substitutions can be used. To introduce deuterium at one or more positions of R₈-R₁₂, compound 18 with the corresponding deuterium substitutions can be used.

Deuterium can be incorporated to various positions having an exchangeable proton, such as the amine N—H and oxindole N—H, via proton-deuterium equilibrium exchange. For example, to introduce deuterium at R₇ or R₁₃, these protons may be replaced with deuterium selectively or non-selectively through a proton-deuterium exchange method known in the art.

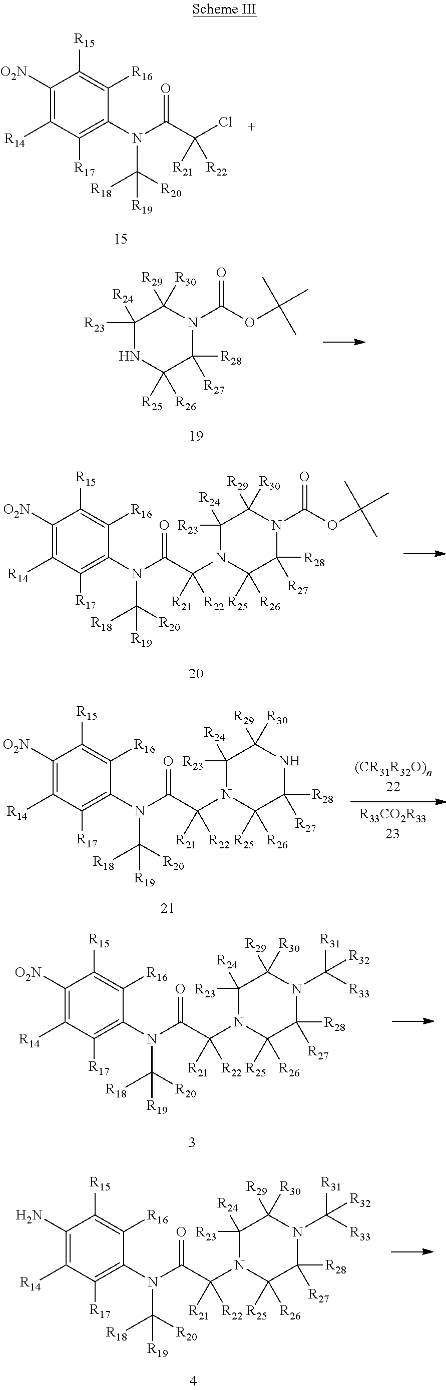

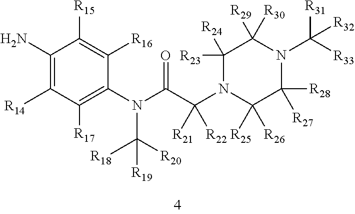

Compound 15 is reacted with compound 19 in the presence of an appropriate base, such as potassium carbonate, in an appropriate solvent, such as acetone, to give compound 20. Compound 20 is treated with an appropriate deprotecting agent, such as trifluoroacetic acid, in an appropriate solvent, such as dichloromethane, to give compound 21. Compound 21 is treated with an appropriate methylating agent, such as a combination of compound 22 and compound 23, to give compound 3. Compound 3 is treated with an appropriate reducing agent, such as a combination of hydrogen gas and an appropriate catalyst, such as palladium on carbon, in an appropriate solvent, such as methanol, to give compound 4. Compound 4 is optionally reacted with an appropriate base, such as potassium carbonate, in the presence of an appropriate protic solvent, such as methanol or deuterated methanol, to give compound 4 wherein hydrogen-deuterium exchange is effected at the positions R₂₁-R₂₂.

Deuterium can be incorporated to different positions synthetically, according to the synthetic procedures as shown in Scheme III, by using appropriate deuterated intermediates. For example, to introduce deuterium at one or more positions of R₁₄-R₂₂, compound 15 with the corresponding deuterium substitutions can be used. To introduce deuterium at one or more positions of R₂₃-R₃₀, compound 19 with the corresponding deuterium substitutions can be used. To introduce deuterium at one or more positions of R₃₁-R₃₃, compound 22 and compound 23 with the corresponding deuterium substitutions can be used.

Deuterium can be incorporated to various positions having an exchangeable proton, such as the carbonyl alpha protons, via proton-deuterium equilibrium exchange. For example, to introduce deuterium at R₂₁-R₂₂, these protons may be replaced with deuterium selectively or non-selectively through a proton-deuterium exchange method known in the art.

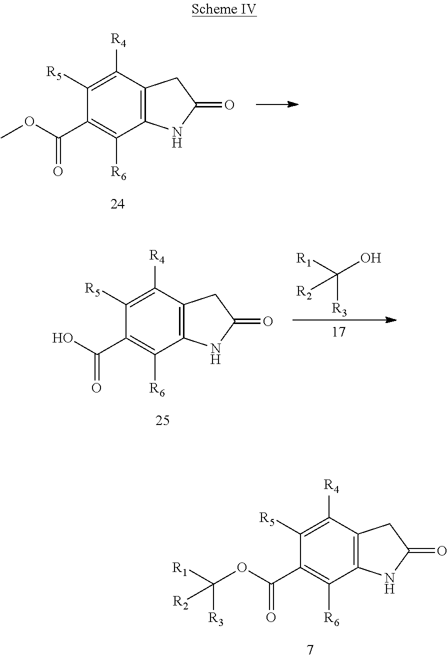

Compound 24 is reacted with an appropriate base, such as sodium hydroxide, in an appropriate solvent, such as a mixture of water and methanol, to give compound 25. Compound 25 is reacted with compound 17 in the presence of an appropriate acid, such as sulfuric acid, to give compound 7.

Deuterium can be incorporated to different positions synthetically, according to the synthetic procedures as shown in Scheme IV, by using appropriate deuterated intermediates. For example, to introduce deuterium at one or more positions of R₄-R₆, compound 24 with the corresponding deuterium substitutions can be used. To introduce deuterium at one or more positions of R₁-R₃, compound 17 with the corresponding deuterium substitutions can be used.

The invention is further illustrated by the following examples. All IUPAC names were generated using CambridgeSoft's ChemDraw 10.0.

### EXAMPLE 1

### (Z)-methyl-3-((4-(N-methyl-2-(4-methylpiperazin-1-yl)acetamido)-phenylamino)(phenyl)methylene)-2-oxoindoline-6-carboxylate (Nintedanib)

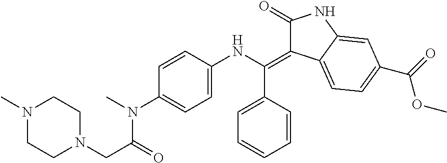

### Step 1

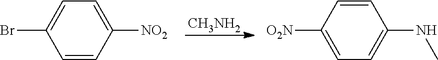

### N-methyl-4-nitrobenzenamine

1-bromo-4-nitro-benzene (5 g, 24.8 mmol) was added to excess aqueous methylamine solution (30%, 30 mL) and heated in a sealed tube for 16 hours. The reaction was cooled to room temperature and the solids were filtered off. The filtrate was evaporated to dryness and the combined solids were purified by trituration with 20 ml pentane to afford methyl-(4-nitro-phenyl)-amine (4.5 g).

### Step 2

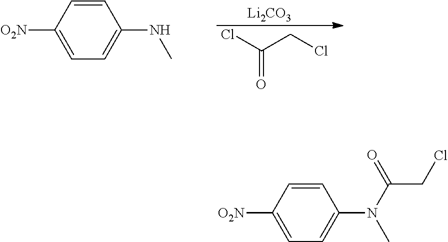

### 2-chloro-N-methyl-N-(4-nitrophenyl) acetamide

23.5 g (0.15 mol) N-methyl-4-nitroaniline was dissolved in 400 ml of dioxane and combined with 22.2 g (0.3 mol) of lithium carbonate. Then 32.2 g (0.18 mol) of chloroacetylchloride was added dropwise such that the internal temperature does not exceed 33° C. After stirring the reaction solution for 3 hours the solution was concentrated to 100 ml, combined with 500 ml of water, and stirred for 1 hour. The precipitate formed was suction filtered, washed with 20 ml water, and dried. The crude product was stirred in 400 ml of ethyl acetate at 40° C. Then the insoluble matter was filtered off, the solution was evaporated to dryness, and the solid residue is triturated with ether (40 ml×2). Yield: 23 g.

### Step 3

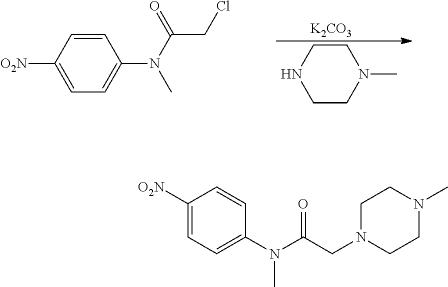

### N-Methyl-2-(4-methylpiperazin-1-yl)-N-(4-nitrophenyl) acetamide

1-Methylpiperazine (7.2 mL, 65 mmol) and potassium carbonate (13.8 g, 100 mmol) were dissolved in acetone (200 mL), and 2-chloro-N-methyl-N-(4-nitrophenyl) acetamide (11.4 g, 50 mmol) was gradually added. The mixture was stirred for 12 hours at ambient temperature. After that time, the precipitates were filtered off and the solvent was evaporated from the filtrate. The residue was taken up in (50×3 ml) ethyl acetate and extracted with 20 ml water. After drying over sodium sulfate, the solvent was removed by evaporation to give 15 g product. LCMS: m/z=293 (MH)⁺.

### Step 4

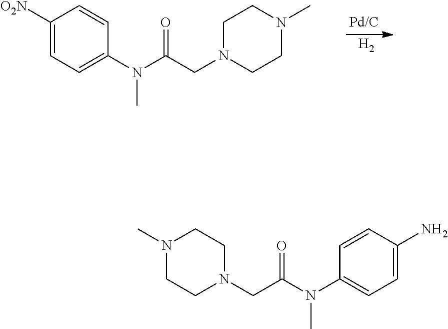

### N-[(4-methyl-piperazin-1-yl)-methylcarbonyl]-N-methyl-p-phenylenediamine

N-Methyl-2-(4-methylpiperazin-1-yl)-N-(4-nitrophenyl) acetamide (5 g, 17 mmol) was dissolved in methanol (50 mL) and hydrogenated (50 psi) at room temperature for 2 hours using 0.6 g 10% palladium on charcoal as catalyst. The catalyst was filtered off and the solvent was removed by evaporation. The residue was triturated with diethyl ether (10 ml×2), filtered, and dried at 80° C. under vacuum to give 3.4 g product. LCMS: m/z=263 (MH)⁺.

### Step 5

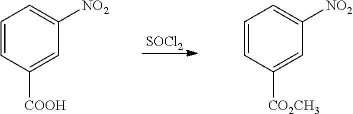

### 3-nitrobenzoic acid methyl ester

3-nitrobenzoic acid (5 g, 19.9 mmol) was dissolved in methanol (50 ml), cooled to 0° C., then SOCl₂ (5.34 g, 44.9 mmol) was dropped in at 0° C. The reaction was then stirred for 2 hours at 50° C. After that time, the precipitates were filtered off to afford 4.5 g of methyl 3-nitrobenzoate. LCMS: m/z=182 (MH)⁺.

### Step 6

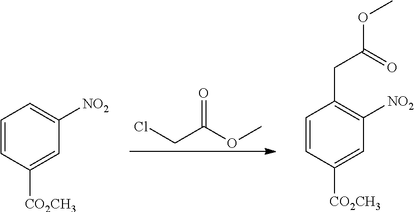

### 4-Methoxycarbonylmethyl-3-nitrobenzoic acid methyl ester

Potassium tert-butylate (5.6 g, 50 mmol) was dissolved in dimethylformamide (50 mL) and a solution of methyl chloroacetate (29.0 ml, 330 mmol) and 3-nitrobenzoic acid methyl ester (4.5 g, 24.8 mmol) in dimethylformamide (10 ml) was slowly added at −10° C. Stirring was continued for 10 min at −10° C. After that time, the mixture was poured into a 0° C. mixture of ice water (1.0 L) and concentrated hydrochloric acid. The precipitate was filtered and washed with water. The residue was recrystallized from 10 ml methanol and dried at 40° C. in vacuum to give 4.2 g of product. LCMS: m/z=254 (MH)⁺.

### Step 7

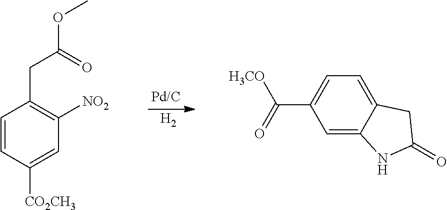 [SMILES: COC(=O)Cc1ccc(C(=O)OC)cc1[N+](=O)[O-] ; "H2" ; COC(=O)c1ccc2c(c1)NC(=O)C2]

### 2-oxo-2,3-Dihydro-1H-indole-6-carboxylic acid methyl ester

4-Methoxycarbonylmethyl-3-nitrobenzoic acid methyl ester (4.2 g, 16.6 mmol) was dissolved in acetic acid (90 ml) and hydrogenated (50 psi) at room temperature for 2.5 hours using 0.6 g 10% palladium on charcoal as catalyst. After that time, the catalyst was filtered off and the solvent was removed by evaporation. The residue was triturated with 5 ml toluene, filtered off, and dried at 100° C. under vacuum to give 3.17 g of product. LCMS: m/z=192 (MH)⁺

### Step 8

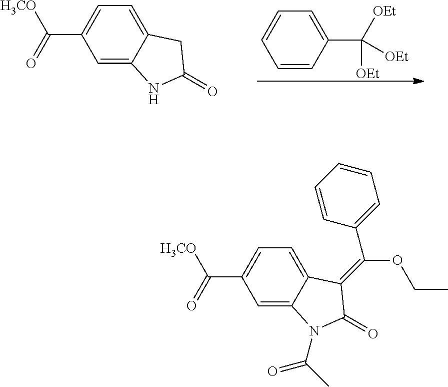 [SMILES: COC(=O)c1ccc2c(c1)NC(=O)C2 ; CCOC(OCC)(OCC)c1ccccc1 ; CCO/C(=C1\C(=O)N(C(C)=O)c2cc(C(=O)OC)ccc21)c1ccccc1]

### 1-acetyl-3-(1-ethoxy-1-phenylmethylene)-6-methoxycarbonyl-2-indolinone

2-oxo-2,3-dihydro-1H-indole-6-carboxylic acid methyl ester (3.17 g, 16.6 mmol) and orthobenzoic acid triethyl ester (11.1 g, 49.8 mmol) was suspended in acetic anhydride (15 mL) and toluene (15 mL). the mixture was stirred at 110° C. overnight. After that time, the solvent was removed by evaporation. The residue was triturated with 10 ml petroleum ether, filtered off, and dried at 50° C. under vacuum to give 6 g product. LCMS: m/z=366 (MH)⁺

### Step 9

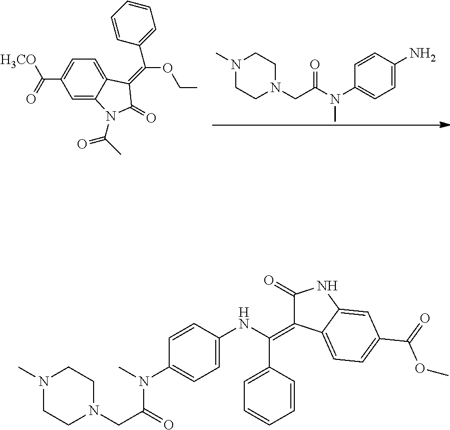

### (Z)-methyl 3-((4-(N-methyl-2-(4-methylpiperazin-1-yl)acetamido)phenylamino)(phenyl)methylene)-2-oxoindoline-6-carboxylate

1-acetyl-3-(1-ethoxy-1-phenylmethylene)-6-methoxycarbonyl-2-indolinone (1.1 g, 3.07 mmol) and (0.91 g, 3.49 mmol)N-[(4-methyl-piperazin-1-yl)-methylcarbonyl]-N-methyl-p-phenylenediamine are dissolved in 10 ml dimethylformamide and mixed for 1 hour at 80° C. After cooling, 0.8 ml piperidine is added and the reaction is further mixed for 2 hours at room temperature. Water is added, the supernatant is removed by suction, and the precipitate is washed again with a small quantity of water. The residue is suspended in 10 ml methanol, the supernatant is removed by suction, and the remaining residue washed with 2 ml cold water and 2 ml diethyl ether. The resulting product is vacuum dried at 110° C. Yield 1.3 g. LCMS: m/z=540 (MH)⁺.

¹HNMR (300 MHz, CDCl₃), δ 12.20 (1H, s), 11.10 (1H, s), 7.60 (5H, m,), 7.40 (1H, s), 7.20 (3H, m), 6.90-7.00 (2H, d, J=8.7 Hz), 5.80-6.00 (1H, d, J=8.4 Hz), 3.80-3.90 (3H, s), 3.10 (3H, s), 2.70-2.80 (2H, s), 2.20 (11H, s).

### EXAMPLE 2

### d₈-(Z)-methyl-3-((4-(N-methyl-2-(4-methylpiperazin-1-yl)acetamido)-phenylamino)(phenyl)methylene)-2-oxoindoline-6-carboxylate (Nintedanib)

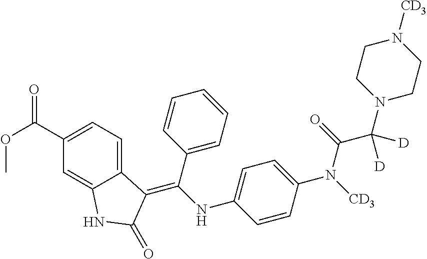 [SMILES: [2H]C([2H])([2H])N1CCN(C([2H])([2H])C(=O)N(c2ccc(N/C(=C3\C(=O)Nc4cc(C(=O)OC)ccc43)c3ccccc3)cc2)C([2H])([2H])[2H])CC1]

### Step 1

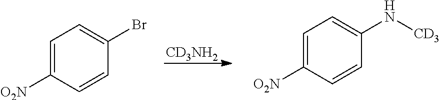

### d₃-N-methyl-4-nitrobenzenamine

Bromo-4-nitro-benzene (5 g, 24.8 mmol) was added to excess aqueous d₃-methylamine solution (30%, 30 ml) and heated to 100° C. in a sealed tube for 16 hours. The reaction was cooled to room temperature and the solids were filtered off. The filtrate was evaporated to dryness and purified by trituration with 20 ml pentane to afford d₃-methyl-(4-nitro-phenyl)-amine (4.5 g).

### Step 2

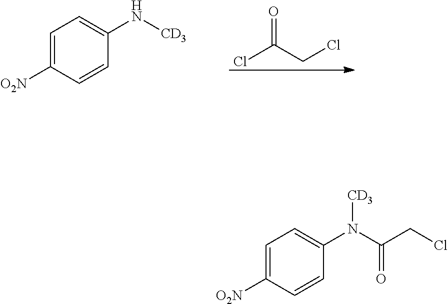 [SMILES: [2H]C([2H])([2H])Nc1ccc([N+](=O)[O-])cc1 ; O=C(Cl)CCl ; [2H]C([2H])([2H])N(C(=O)CCl)c1ccc([N+](=O)[O-])cc1]

### d₃-2-chloro-N-methyl-N-(4-nitrophenyl) acetamide

d₃-N-methyl-4-nitroaniline (23.5 g, 0.15 mol, 1.00 equiv) was dissolved in 400 ml of dioxane and combined with lithium carbonate (22.2 g, 0.3 mol, 2.00 equiv). Then chloroacetylchloride (32.2 g, 0.18 mol, 1.20 equiv) was added dropwise such that the internal temperature did not exceed 33° C. After stifling the reaction solution for 3 hours the solution was evaporated to a volume of 100 ml, combined with 500 ml of water and stirred for 1 hour. The precipitate formed was filtered, washed with 20 ml water, and dried. The crude product was stirred in 400 ml of ethyl acetate at 40° C. The insoluble matter was filtered off, the solution was evaporated, and the solid residue was triturated with ether (40 ml×2). This resulted in 23 g (67.7%) of d₃-2-chloro-N-methyl-N-(4-nitrophenyl) acetamide as yellow solid.

### Step 3

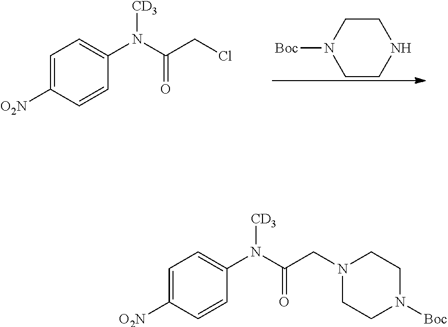 [SMILES: [2H]C([2H])([2H])N(C(=O)CCl)c1ccc([N+](=O)[O-])cc1 ; CC(C)(C)OC(=O)N1CCNCC1 ; [2H]C([2H])([2H])N(C(=O)CN1CCN(C(=O)OC(C)(C)C)CC1)c1ccc([N+](=O)[O-])cc1]

### d₃-N-Methyl-2-(4-tert-butoxycarbonylpiperazin-1-yl)-N-(4-nitrophenyl) acetamide

N-tert-butoxycarbonyl-piperazine (7.2 mL, 65 mmol, 1.3 equiv) and potassium carbonate (13.8 g, 100 mmol, 2.00 equiv) were dissolved in acetone (200 mL), and d₃-2-chloro-N-methyl-N-(4-nitrophenyl) acetamide (11.55 g, 50 mmol, 1.00 equiv) was gradually added. The mixture was stirred for 12 hours at ambient temperature. After that time, the precipitates were filtered off and the solvent was evaporated from the filtrate. The residue was taken up in (50 ml×3) ethyl acetate and extracted with 20 ml water. After drying over sodium sulfate, the solvent was evaporated to give 15.2 g (80%) product. LCMS: m/z=382 (MH)⁺.

### Step 4

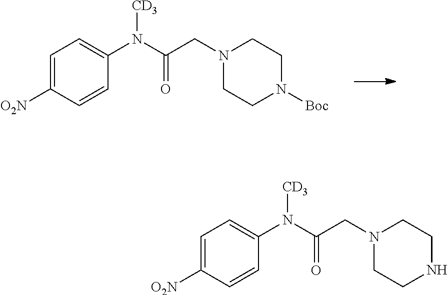 [SMILES: [2H]C([2H])([2H])N(C(=O)CN1CCN(C(=O)OC(C)(C)C)CC1)c1ccc([N+](=O)[O-])cc1 ; [2H]C([2H])([2H])N(C(=O)CN1CCNCC1)c1ccc([N+](=O)[O-])cc1]

### d₃-N-methyl-N-(4-nitrophenyl)-2-(piperazin-1-yl)acetamide

To a solution of d₃-N-Methyl-2-(4-tert-butoxycarbonylpiperazin-1-yl)-N-(4-nitrophenyl) acetamide (8.7 g, 22.8 mmol, 1 equiv) in dichloromethane (20 ml), was gradually added CF₃COOH (15.6 g, 137 mmol, 6 equiv). After stirring the reaction solution for 3 hours at 40° C. the solution was evaporated. Then 50 ml H₂O was added, the pH was adjusted to 8 with Na₂CO₃. The resulting solution was extracted with 3×200 ml of dichloromethane and the organic layers combined and dried over anhydrous sodium sulfate. The solids were filtered. The resulting mixture was concentrated under vacuum. This resulted in 5 g (77.6%) of d₃-N-methyl-N-(4-nitrophenyl)-2-(piperazin-1-yl)acetamide as a yellow solid. LC-MS: m/z=282 (M+H)⁺.

### Step 5

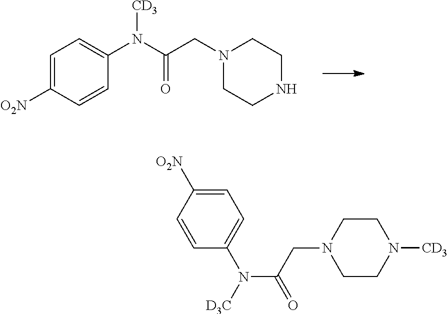 [SMILES: [2H]C([2H])([2H])N(C(=O)CN1CCNCC1)c1ccc([N+](=O)[O-])cc1 ; [2H]C([2H])([2H])N1CCN(CC(=O)N(c2ccc([N+](=O)[O-])cc2)C([2H])([2H])[2H])CC1]

### d₆-N-methyl-2-(4-methylpiperazin-1-yl)-N-(4-nitrophenyl)acetamide

d₃-N-methyl-N-(4-nitrophenyl)-2-(piperazin-1-yl)acetamide (5 g, 17.8 mmol, 1 equiv) and d-paraformaldehyde (1.14 g, 35.6 mmol, 2.00 equiv) were dissolved in DCOOD (4.3 g, 89 mmol, 5 equiv). The mixture was stirred for 12 hours at reflux. The pH of the solution was adjusted to 8 and extracted with (50 ml×3) ethyl acetate. After drying over sodium sulfate, the solvent was removed by evaporation to give 5.0 g (94%) product. LCMS: m/z=299 (MH)⁺.

### Step 6

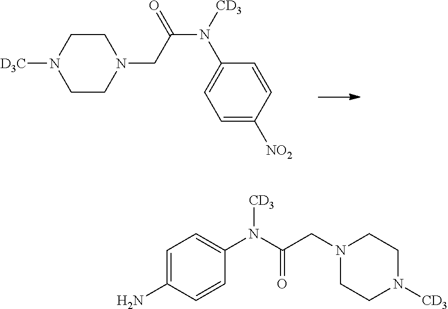 [SMILES: [2H]C([2H])([2H])N1CCN(CC(=O)N(c2ccc([N+](=O)[O-])cc2)C([2H])([2H])[2H])CC1 ; [2H]C([2H])([2H])N1CCN(CC(=O)N(c2ccc(N)cc2)C([2H])([2H])[2H])CC1]

### d₆-N-(4-aminophenyl)-N-methyl-2-(4-methylpiperazin-1-yl)acetamide

d₆-N-Methyl-2-(4-methylpiperazin-1-yl)-N-(4-nitrophenyl) acetamide (5 g, 16.8 mmol) was dissolved in methanol (50 mL) and hydrogenated (50 psi) at room temperature for 2 hours using 0.6 g 10% palladium on charcoal as catalyst. After that time, the catalyst was filtered off and the solvent was removed by evaporation. The residue was triturated with diethyl ether (20 ml×2), filtered, and dried under vacuum to give 3.4 g (75%) of d₆-N-(4-aminophenyl)-N-methyl-2-(4-methylpiperazin-1-yl)acetamide as a yellow solid. LCMS: m/z=269 (MH)⁺.

### Step 8

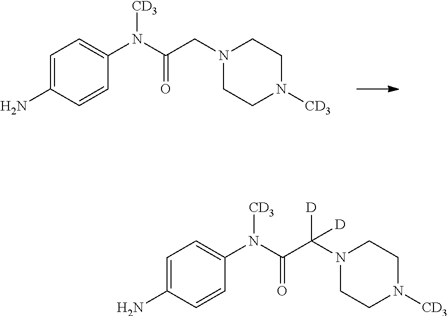 [SMILES: [2H]C([2H])([2H])N1CCN(CC(=O)N(c2ccc(N)cc2)C([2H])([2H])[2H])CC1 ; [2H]C([2H])([2H])N1CCN(C([2H])([2H])C(=O)N(c2ccc(N)cc2)C([2H])([2H])[2H])CC1]

### d₈-N-(4-aminophenyl)-N-methyl-2-(4-methylpiperazin-1-yl)acetamide

Into a sealed tube was added d₆-N-(4-aminophenyl)-N-methyl-2-(4-methylpiperazin-1-yl)acetamide (2.68 g, 10 mmol, 1 equiv), K2CO₃ (2.76 g, 20 mmol, 2 equiv), and 30 ml of CD₃OD. The resulting solution was stirred overnight at 80° C. After that time, the solids were filtered off and washed with 30 ml of ethyl acetate, and the solvent was removed by evaporation. 2 g (73%) of d₈-N-(4-aminophenyl)-N-methyl-2-(4-methylpiperazin-1-yl)acetamide was obtained. The product was used in the next reaction without further purification. LCMS: m/z=271 (MH)⁺.

### Step 9

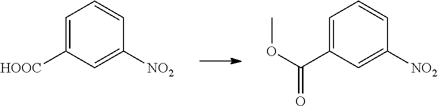

### 3-nitrobenzoic acid methyl ester

3-nitrobenzoic acid (5 g, 19.9 mmol) was dissolved in methanol (50 ml), cooled to 0° C., and SOCl₂ (5.34 g, 44.9 mmol) was added dropwise at 0° C. The reaction was then stirred for 2 hours at 50° C. After that time, the precipitates were filtered off to afford 4.5 g of methyl 3-nitrobenzoate. LCMS: m/z=182 (MH)⁺.

### Step 10

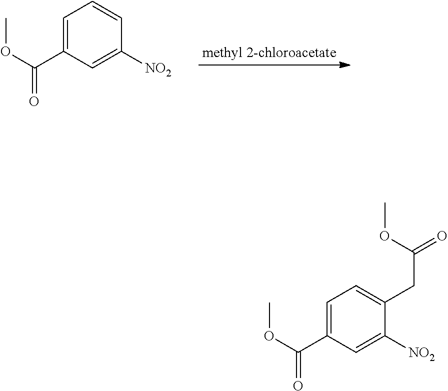 [SMILES: COC(=O)c1cccc([N+](=O)[O-])c1 ; COC(=O)Cc1ccc(C(=O)OC)cc1[N+](=O)[O-]]

### 4-Methoxycarbonylmethyl-3-nitrobenzoic acid methyl ester

Potassium tert-butylate (5.6 g, 50 mmol) was dissolved in dimethylformamide (50 ml) and a solution of methyl chloroacetate (29.0 mL, 330 mmol) and 3-nitrobenzoic acid methyl ester (4.5 g, 24.8 mmoL) in dimethylformamide (10 ml) was slowly added at −10° C. Stirring was continued for 10 min at −10° C. After that time, the mixture was poured into a 0° C. mixture of ice water (1.0 L) and concentrated hydrochloric acid. The precipitate was filtered off and washed with water. The residue was recrystallized from 10 ml methanol and dried at 40° C. in vacuum to give 4.2 g of product. LCMS: m/z=254 (MH)⁺.

### Step 11

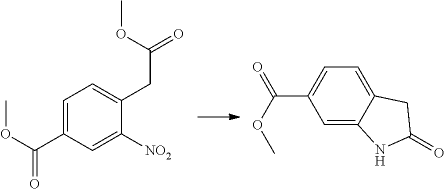 [SMILES: COC(=O)Cc1ccc(C(=O)OC)cc1[N+](=O)[O-] ; COC(=O)c1ccc2c(c1)NC(=O)C2]

### 2-oxo-2,3-Dihydro-1H-indole-6-carboxylic acid methyl ester

4-Methoxycarbonylmethyl-3-nitrobenzoic acid methyl ester (4.2 g, 16.6 mmol) was dissolved in acetic acid (90 mL) and hydrogenated (50 psi) at room temperature for 2.5 h using 0.6 g 10% palladium on charcoal as catalyst. After that time, the catalyst was filtered off and the solvent was removed by evaporation. The residue was triturated with 5 ml toluene, filtered off, and dried at 100° C. under vacuum to give 3.17 g of product. LCMS: m/z=192 (MH)⁺.

### Step 12

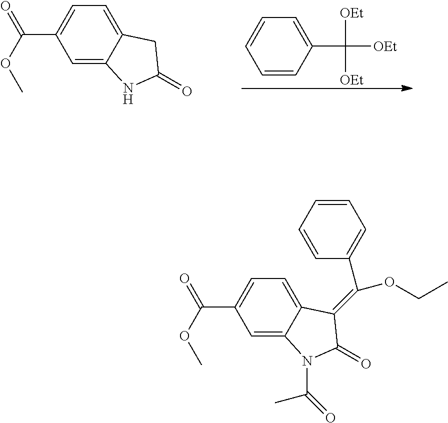 [SMILES: COC(=O)c1ccc2c(c1)NC(=O)C2 ; CCOC(OCC)(OCC)c1ccccc1 ; CCO/C(=C1\C(=O)N(C(C)=O)c2cc(C(=O)OC)ccc21)c1ccccc1]

### 1-acetyl-3-(1-ethoxy-1-phenylmethylene)-6-methoxycarbonyl-2-indolinone

2-oxo-2,3-Dihydro-1H-indole-6-carboxylic acid methyl ester (3.17 g, 16.6 mmol) and orthobenzoic acid triethyl ester (11.1 g, 49.8 mmol) was suspended in acetic anhydride (15 mL) and toluene (15 mL). The mixture was stirred at 110° C. overnight. After that time, the solvent was removed by evaporation. The residue was triturated with 10 ml petroleum ether, filtered off, and dried at 50° C. under vacuum to give 6 g product. LCMS: m/z=366 (MH)⁺.

### Step 13

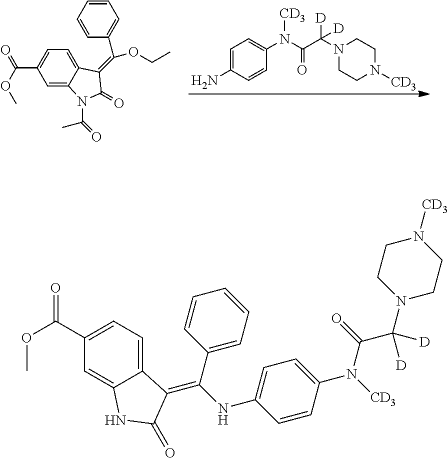 [SMILES: CCO/C(=C1\C(=O)N(C(C)=O)c2cc(C(=O)OC)ccc21)c1ccccc1 ; [2H]C([2H])([2H])N1CCN(C([2H])([2H])C(=O)N(c2ccc(N)cc2)C([2H])([2H])[2H])CC1 ; [2H]C([2H])([2H])N1CCN(C([2H])([2H])C(=O)N(c2ccc(N/C(=C3\C(=O)Nc4cc(C(=O)OC)ccc43)c3ccccc3)cc2)C([2H])([2H])[2H])CC1]

### d₈-(Z)-methyl 3-((4-(N-methyl-2-(4-methylpiperazin-1-yl)acetamido)phenylamino) (phenyl)methylene)-2-oxoindoline-6-carboxylate

1-acetyl-3-(1-ethoxy-1-phenylmethylene)-6-methoxycarbonyl-2-indolinone (1.1 g, 3.07 mmol, 1 equiv) and d₈-N-[(4-methyl-piperazin-1-yl)-methylcarbonyl]-N-methyl-p-phenylenediamine (0.91 g, 3.49 mmol, 1.15 equiv) are dissolved in 10 ml dimethylformamide and stirred for 1 hour at 80° C. After cooling, 0.8 ml piperidine is added and the reaction is stirred for 2 hours at room temperature. Water is added, the supernatant is removed by suction, and the precipitate is washed again with a small quantity of water. The residue is suspended in 10 ml methanol, the supernatant is removed by suction, and the remaining residue washed with 2 ml cold water and 2 ml diethyl ether. The resulting product is vacuum dried at 110° C. This resulted in 1.3 g of d₈-(Z)-methyl 3-((4-(N-methyl-2-(4-methylpiperazin-1-yl)acetamido)phenylamino) (phenyl)methylene)-2-oxoindoline-6-carboxylate as a white solid. LCMS: m/z=548 (MH)⁺.

¹H-NMR (300 MHz, CDCl₃), δ 12.23 (1H, s), 10.96 (1H, s), 7.61-7.49 (5H, m), 7.42 (1H, d), 7.21-6.90 (3H, m), 6.91-6.88 (2H, d, J=8.7 Hz), 5.84-5.82 (1H, d, J=8.1 Hz), 3.77 (3H, s), 1.99 (8H, s).

### EXAMPLE 3

### d₁₁-(Z)-methyl-3-((4-(N-methyl-2-(4-methylpiperazin-1-yl)acetamido)-phenylamino)(phenyl)methylene)-2-oxoindoline-6-carboxylate (Nintedanib)

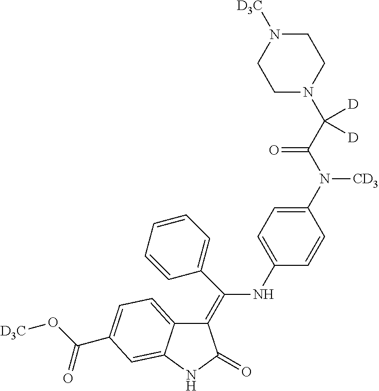 [SMILES: [2H]C([2H])([2H])OC(=O)c1ccc2c(c1)NC(=O)/C2=C(\Nc1ccc(N(C(=O)C([2H])([2H])N2CCN(C([2H])([2H])[2H])CC2)C([2H])([2H])[2H])cc1)c1ccccc1]

### Step 1

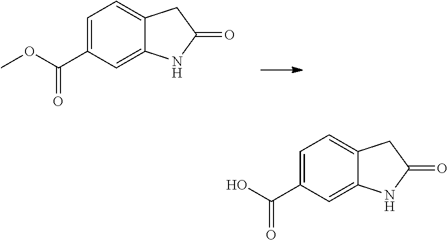

### 2-oxoindoline-6-carboxylic acid

Sodium hydroxide solution (1N, 20 ml) was added to a solution of methyl 2-oxoindoline-6-carboxylate (2 g, 10.46 mmol, 1.00 equiv) in methanol (20 ml). The resulting solution was stirred for 2 hours at 80° C. The reaction mixture was cooled to 30° C., diluted with 50 ml of H₂O and extracted with 2×30 mL of dichloromethane. The aqueous layers were combined and the pH adjusted to 2 with aqueous hydrochloric acid (6 N). The solids were collected by filtration and dried to give the title product 1.28 g (69%) as a brown solid.

¹H NMR (400 MHz, CDCl₃) δ: 12.86 (s, 1H), 10.50 (s, 1H), 7.55 (m, 1H), 7.32-7.30 (m, 2H), 3.56 (s, 2H).

### Step 2

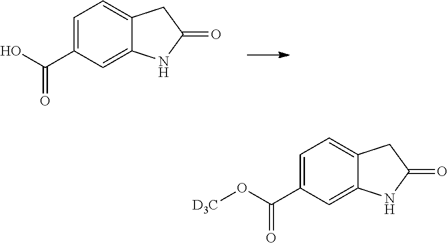 [SMILES: O=C1Cc2ccc(C(=O)O)cc2N1 ; [2H]C([2H])([2H])OC(=O)c1ccc2c(c1)NC(=O)C2]

### d₃-methyl 2-oxoindoline-6-carboxylate

A solution of 2-oxoindoline-6-carboxylic acid (1.2 g, 6.77 mmol, 1.00 equiv), sulfuric acid (98%, catalytic amount) in CD₃OD (50 mL) was stirred for 24 hours at 60° C. The reaction mixture was cooled to room temperature and the filtrate was concentrated under vacuum and poured into ice water. The pH was adjusted to 8 with NaHCO₃ and the aqueous solution was extracted with ethyl acetate. The ethyl acetate was concentrated and the residue applied onto a silica gel column and eluted with ethyl acetate/petroleum ether (1:5) to give the title product 1.0 g (75%) as a brown solid.

¹H NMR (400 MHz, CDCl₃) δ: 10.54 (s, 1H), 10.50 (s, 1H), 7.58 (m, 1H), 7.34 (d, J=7.8 Hz, 2H), 3.45 (s, 2H).

### Step 3

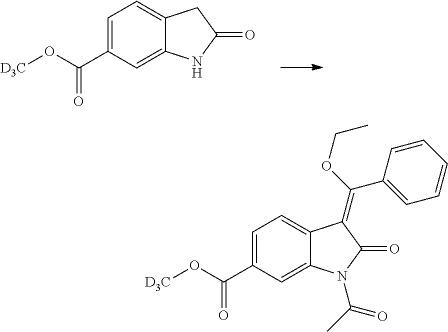 [SMILES: [2H]C([2H])([2H])OC(=O)c1ccc2c(c1)NC(=O)C2 ; [2H]C([2H])([2H])OC(=O)c1ccc2c(c1)N(C(C)=O)C(=O)/C2=C(/OCC)c1ccccc1]

### d₃-(Z)-methyl 1-acetyl-3-(ethoxy(phenyl)methylene)-2-oxoindoline-6-carboxylate

1-(triethoxymethyl)benzene (1.23 g, 5.48 mmol, 2.99 equiv) was added to a solution of d₃-methyl 2-oxoindoline-6-carboxylate (360 mg, 1.83 mmol, 1.00 equiv) in toluene/acetic anhydride (7 ml/7 ml). The resulting solution was stirred for 3.5 hours at 110-115° C. The reaction mixture was cooled to 50° C. and concentrated under vacuum. The residue was applied onto a silica gel column and eluted with ethyl acetate/petroleum ether (1:15) to give the title product 0.44 g (62%) as a yellow solid.

¹H NMR (400 MHz, DMSO-d₆) δ: 8.75 (s, 1H), 8.10 (d, J=8.1 Hz, 1H), 7.88 (m, 1H), 7.49-7.69 (m, 5H), 4.01 (q, J=7.2 Hz, 2H), 2.45 (s, 3H), 1.35 (t, J=7.2 Hz, 3H).

### Step 4

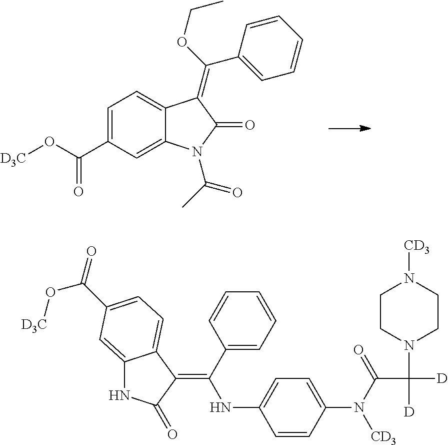 [SMILES: [2H]C([2H])([2H])OC(=O)c1ccc2c(c1)N(C(C)=O)C(=O)/C2=C(/OCC)c1ccccc1 ; [2H]C([2H])([2H])OC(=O)c1ccc2c(c1)NC(=O)/C2=C(\Nc1ccc(N(C(=O)C([2H])([2H])N2CCN(C([2H])([2H])[2H])CC2)C([2H])([2H])[2H])cc1)c1ccccc1]

### d₁₁-(Z)-methyl 3-((4-(N-methyl-2-(4-methylpiperazin-1-yl)acetamido)phenylamino)(phenyl)methylene)-2-oxoindoline-6-carboxylate

d₈-(Z)-methyl 3-((4-(N-methyl-2-(4-methylpiperazin-1-yl)acetamido)phenylamino) (phenyl)methylene)-2-oxoindoline-6-carboxylate (140 mg, 0.52 mmol, 1.00 equiv) and d₃-(Z)-methyl 1-acetyl-3-(ethoxy(phenyl)methylene)-2-oxoindoline-6-carboxylate (180 mg, 0.49 mmol, 0.94 equiv) were dissolved in dimethylformamide (3 ml). The resulting solution was stirred for 1.5 hours at 80° C. The temperature was cooled to 20° C. and piperidine (0.2 mL) was added. The resulting solution was allowed to react, with stifling, for an additional 1.5 h at 30° C. The reaction mixture was cooled to 10° C. and then quenched by the addition of 30 mL of D₂O. The solids were collected by filtration. The residue was dissolved in 10 mL of CD₃OD and concentrated under vacuum. The residue was applied onto a silica gel column and eluted with dichloromethane/MeOH (30:1-10:1) to give the title product 100 mg (37%) as a yellow solid. LC-MS: m/z=551 (MH)⁺.

¹H NMR (400 MHz, DMSO-d₆) δ: 12.23 (s, 1H), 10.98 (s, 1H), 7.60-7.42 (m, 6H), 7.21-7.13 (m, 3H), 6.88 (d, J=8.7 Hz, 2H), 5.83 (d, J=8.1 Hz, 1H), 2.21 (m, 8H).

The following compounds can generally be made using the methods described above. It is expected that these compounds when made will have activity similar to those described in the examples above.

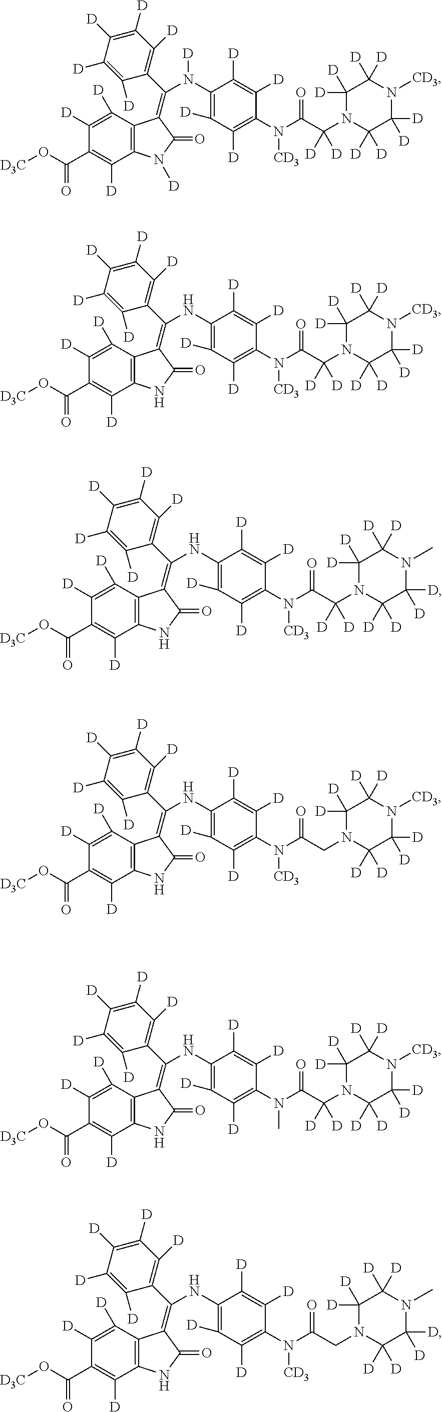

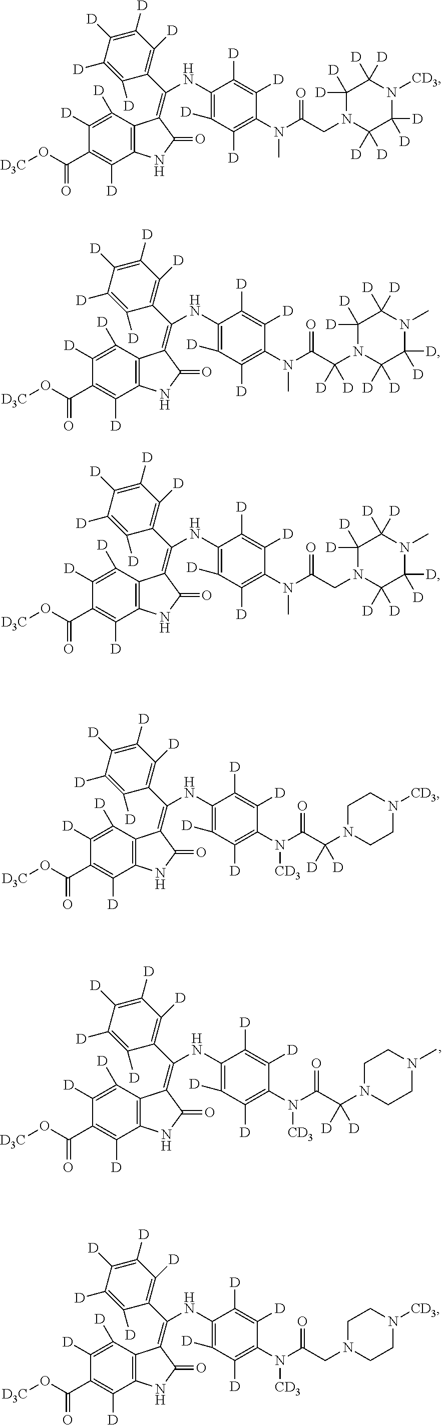

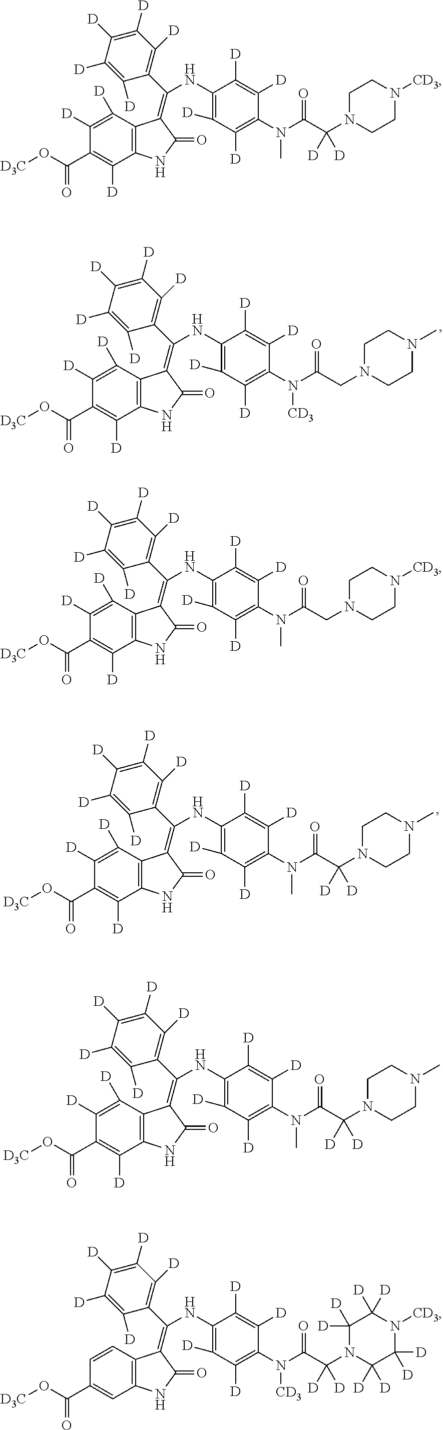

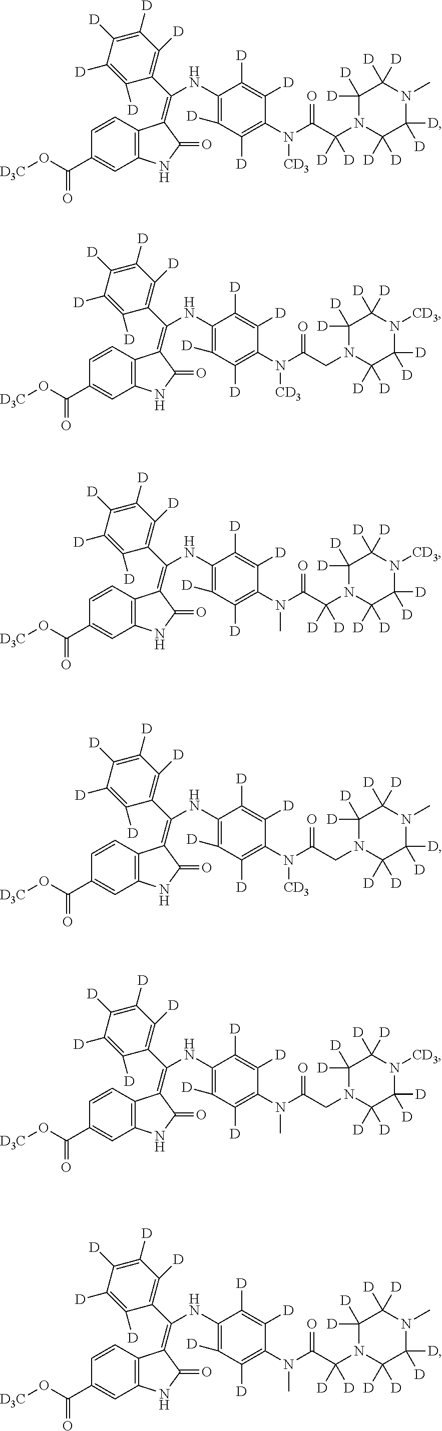

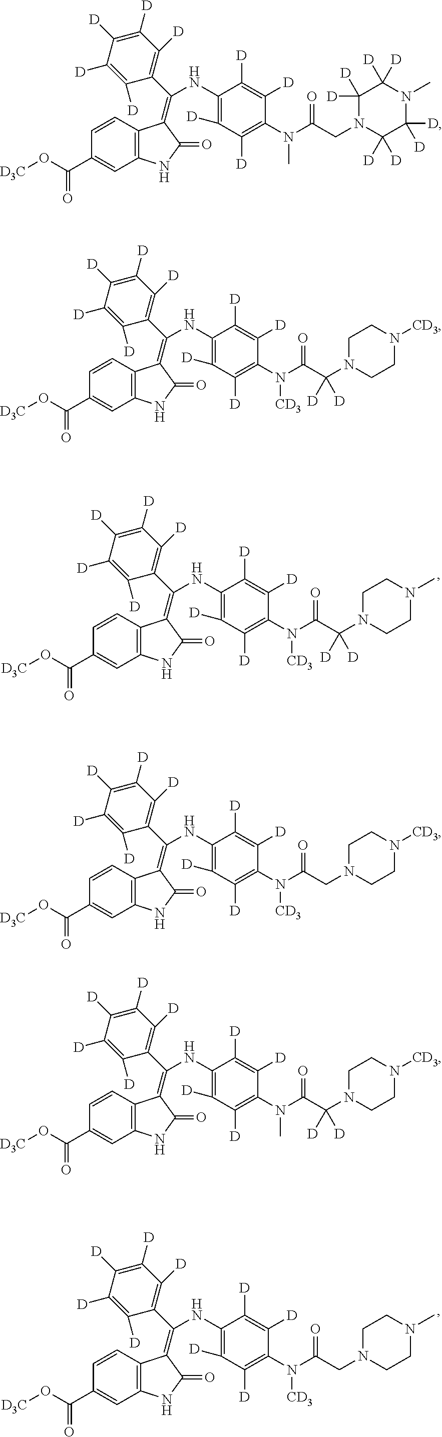

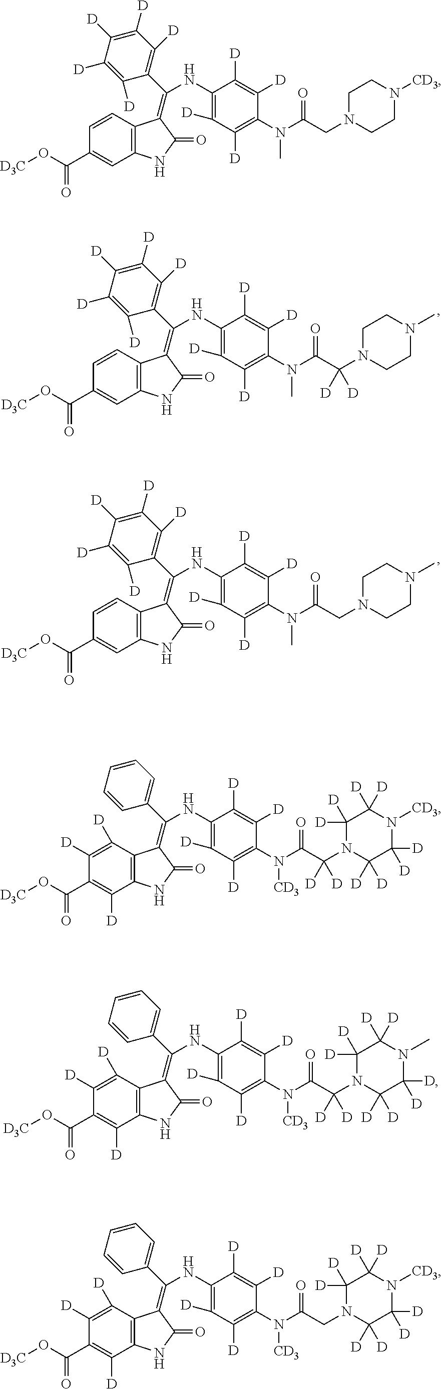

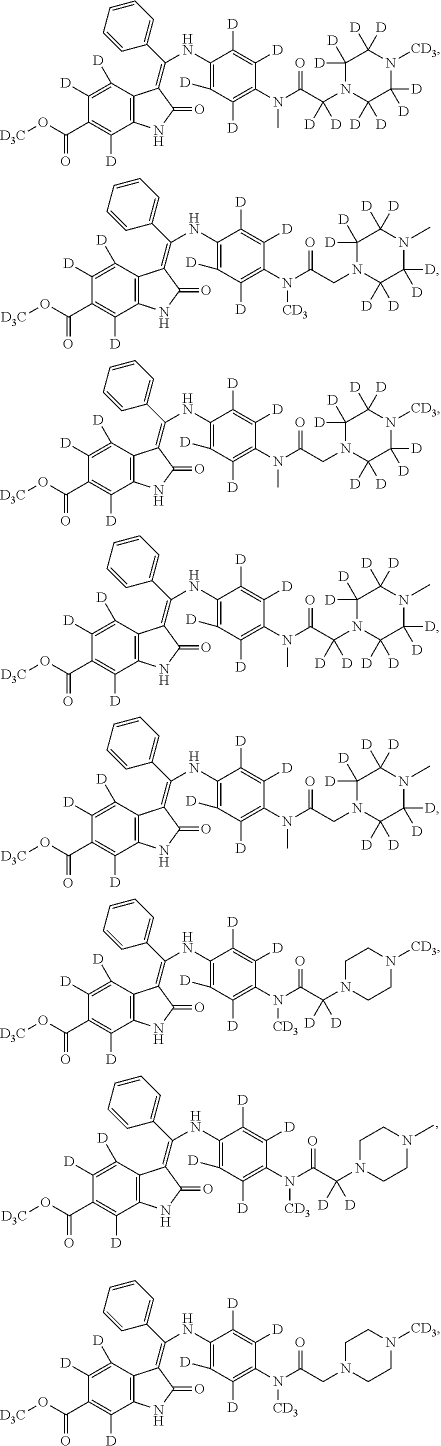

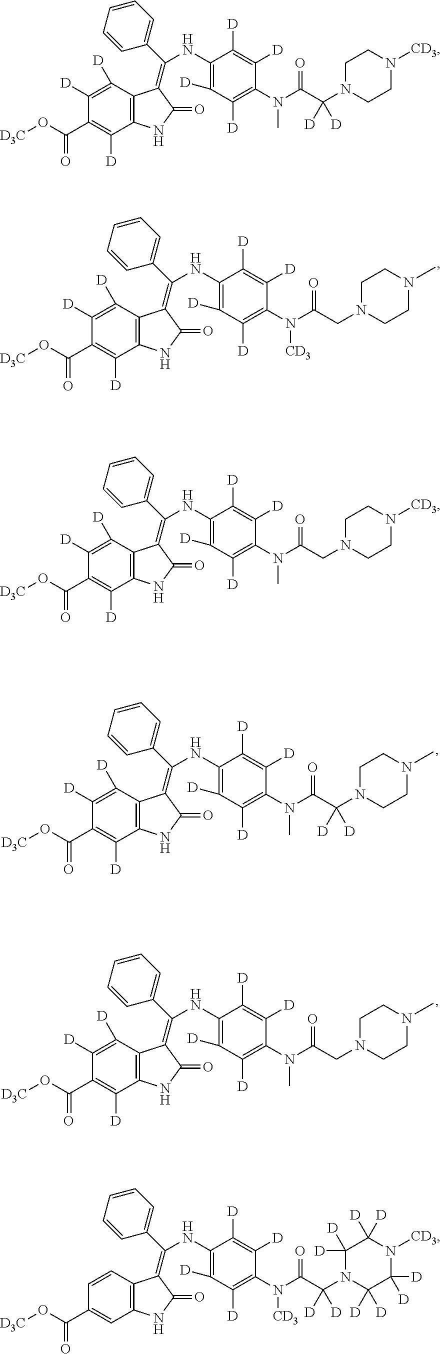

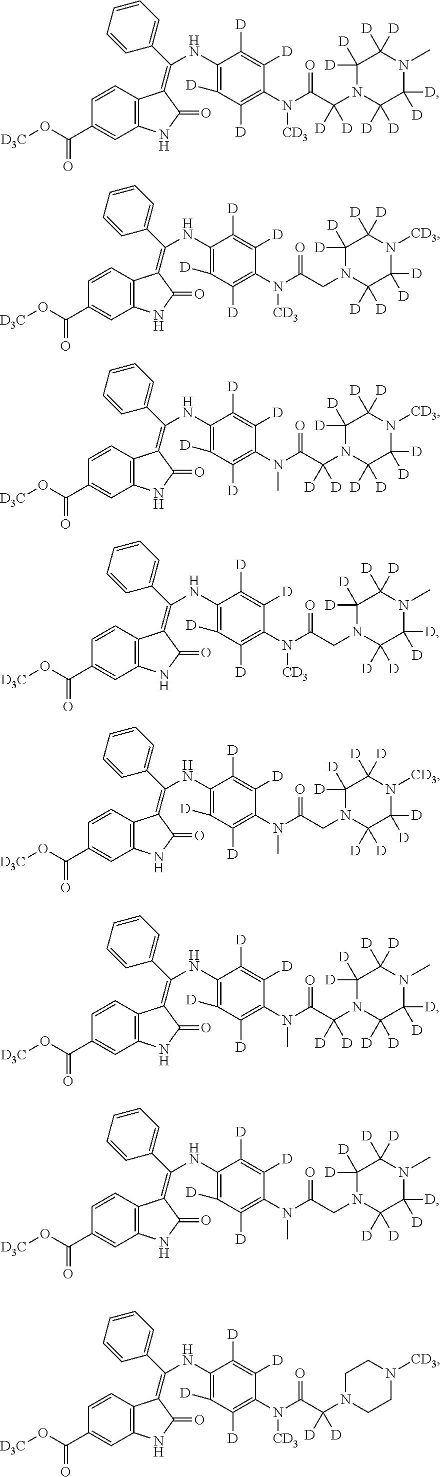

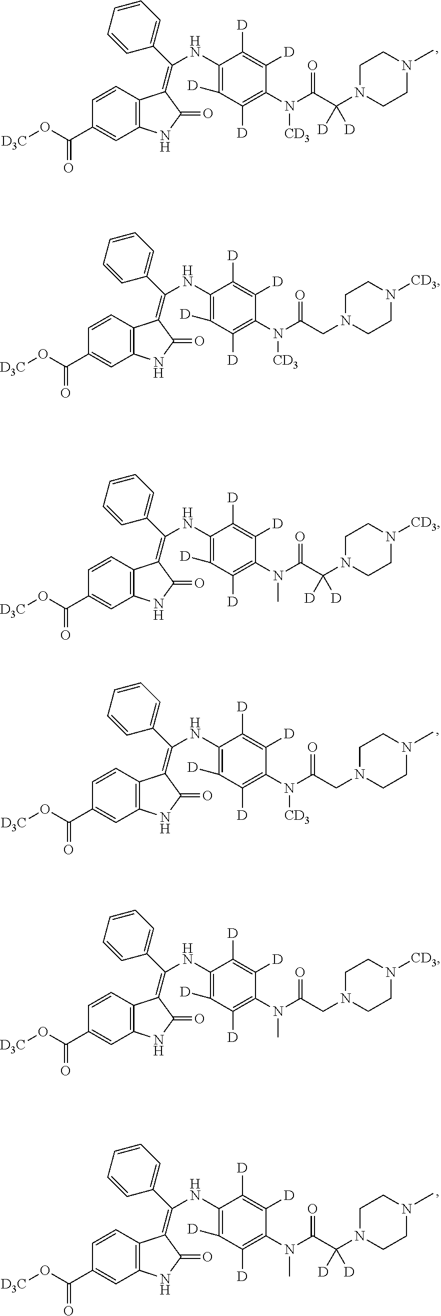

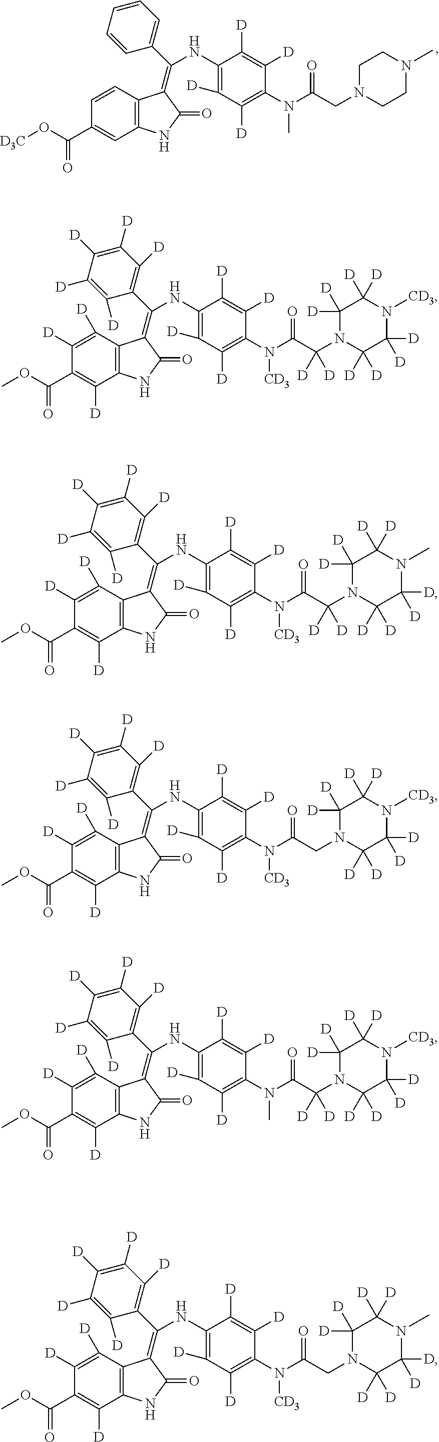

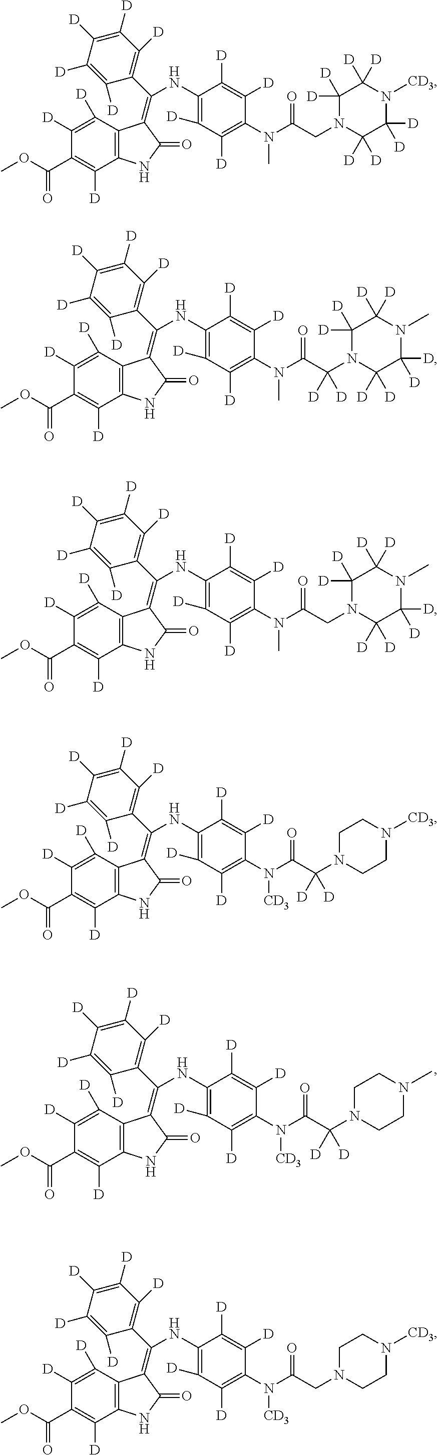

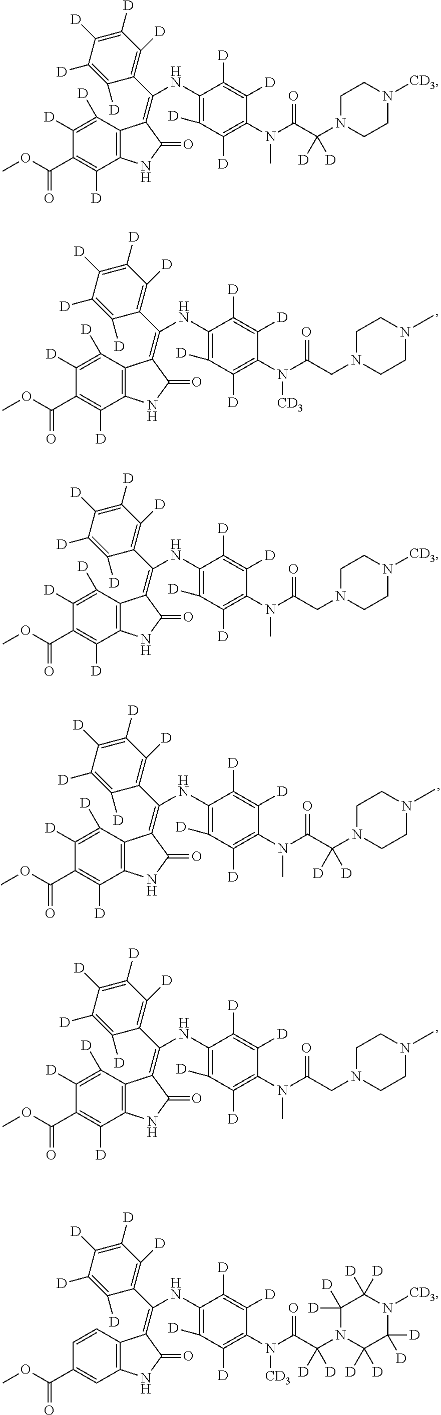

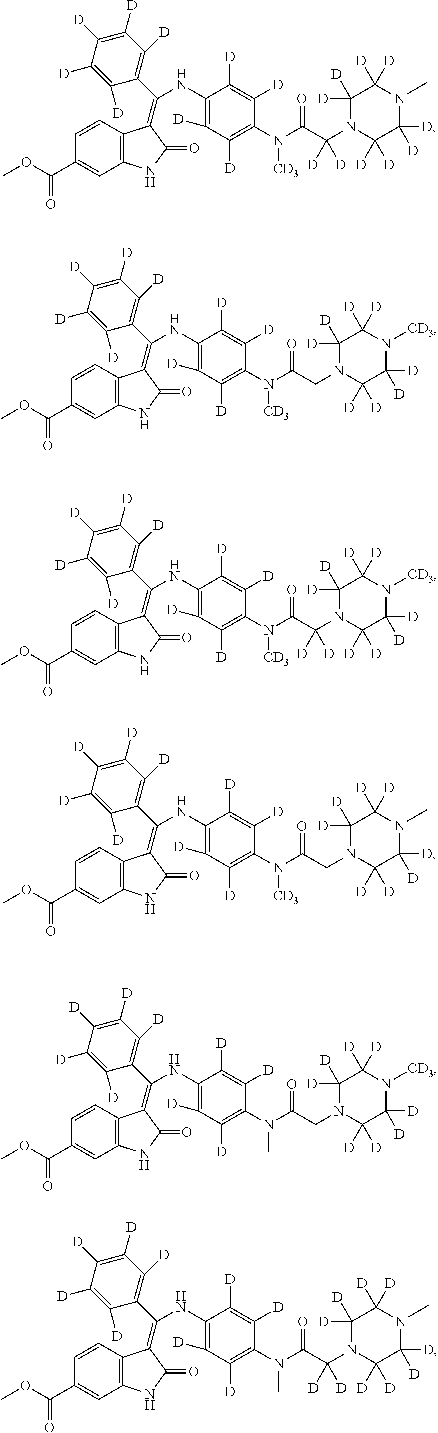

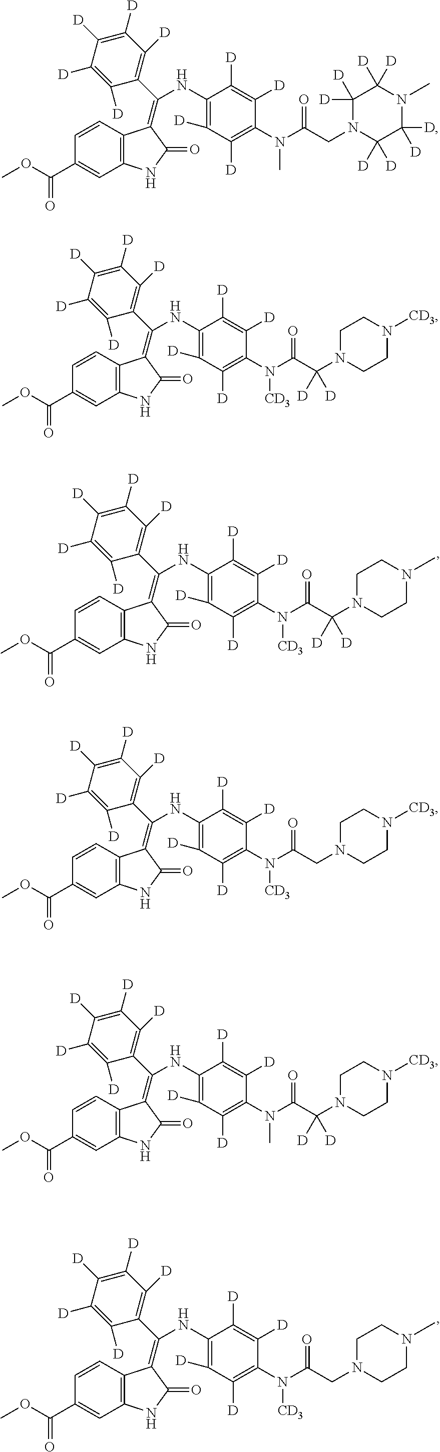

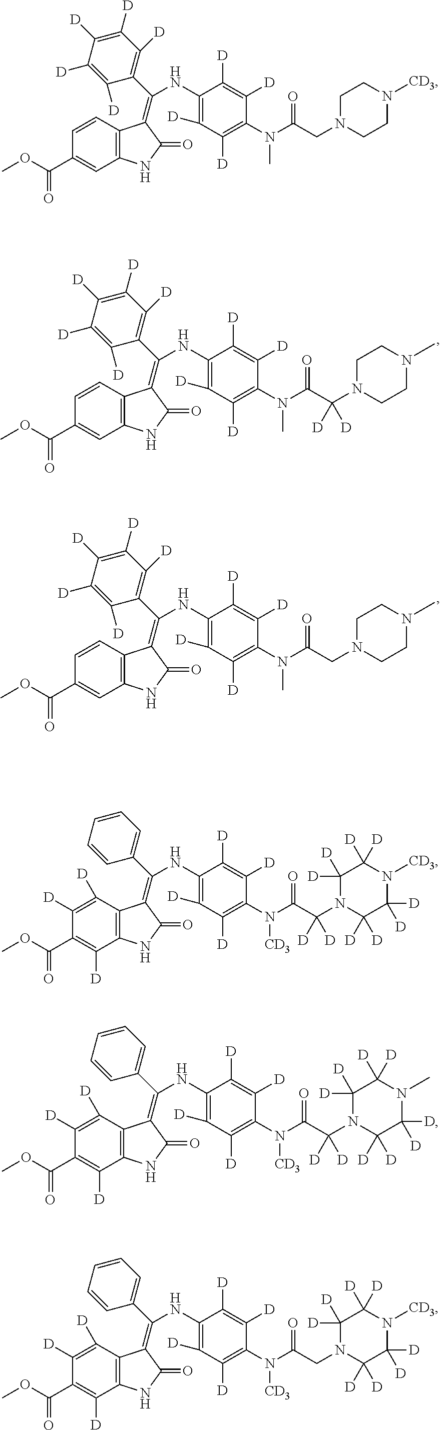

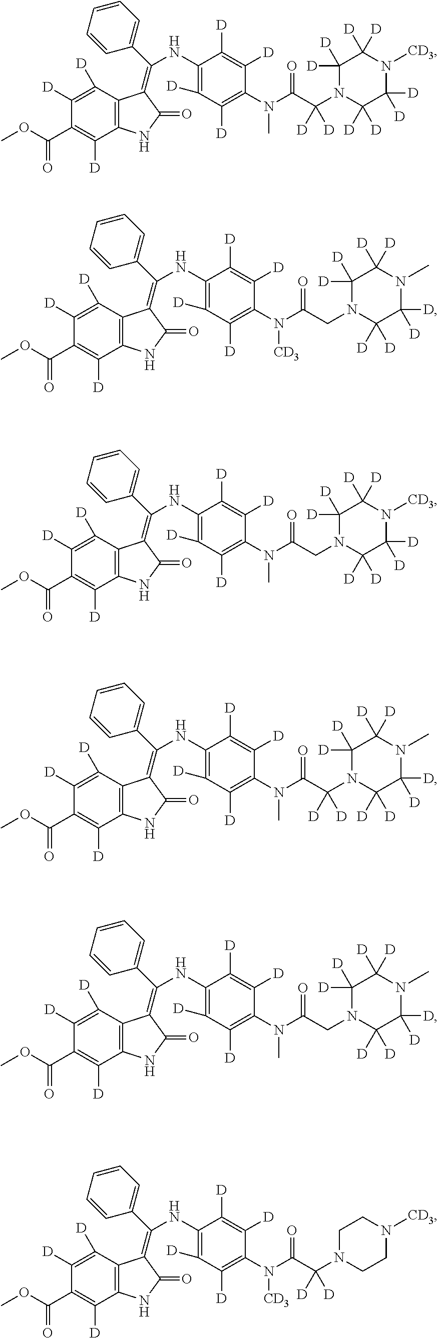

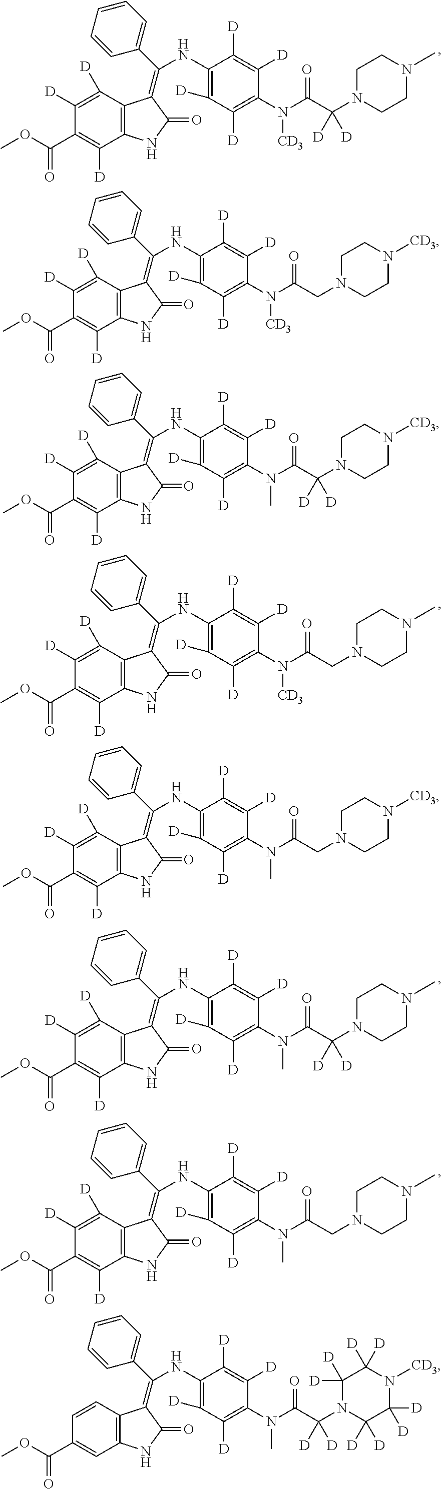

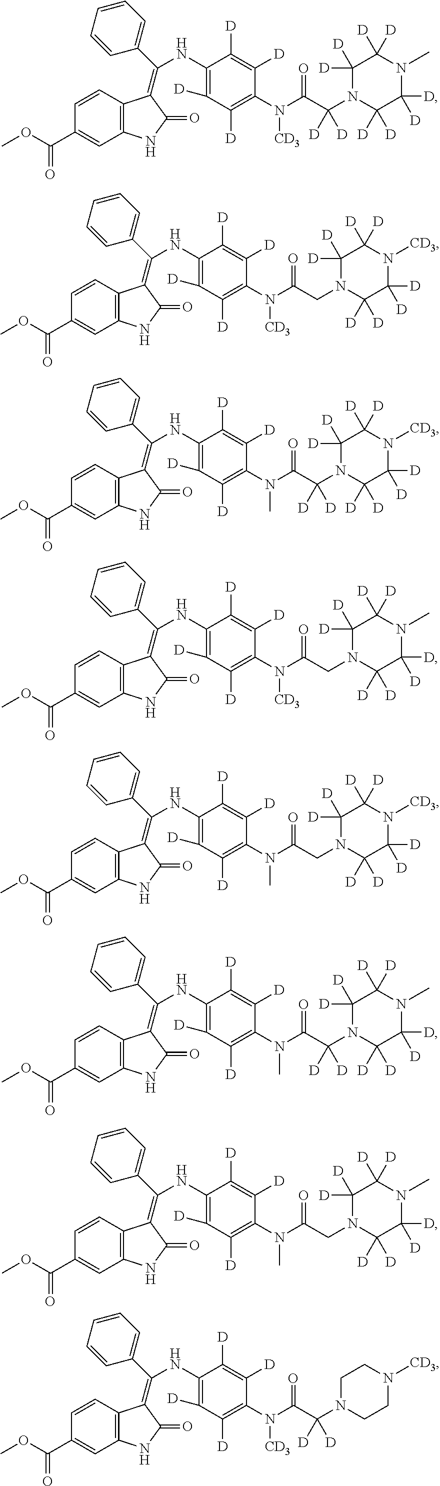

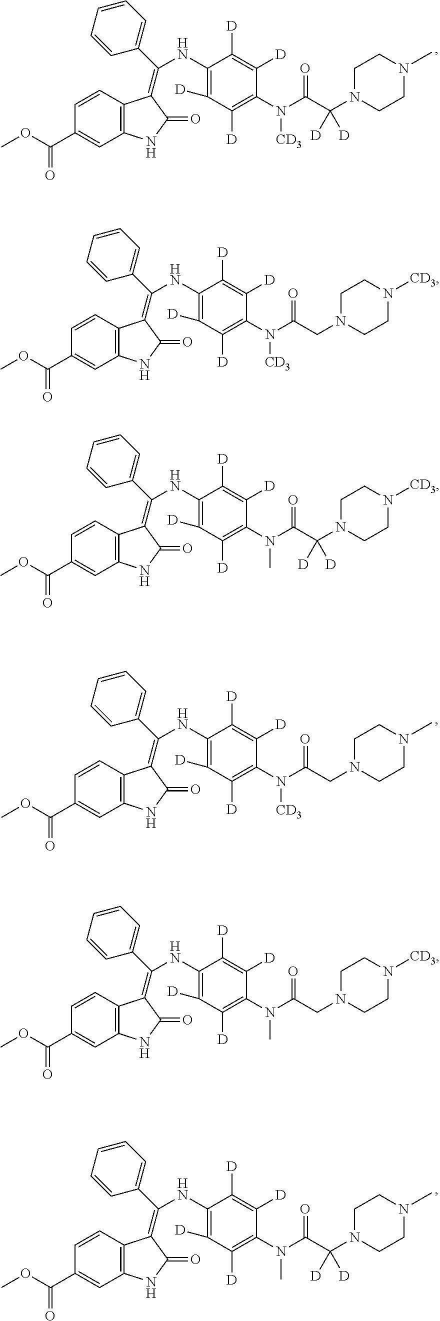

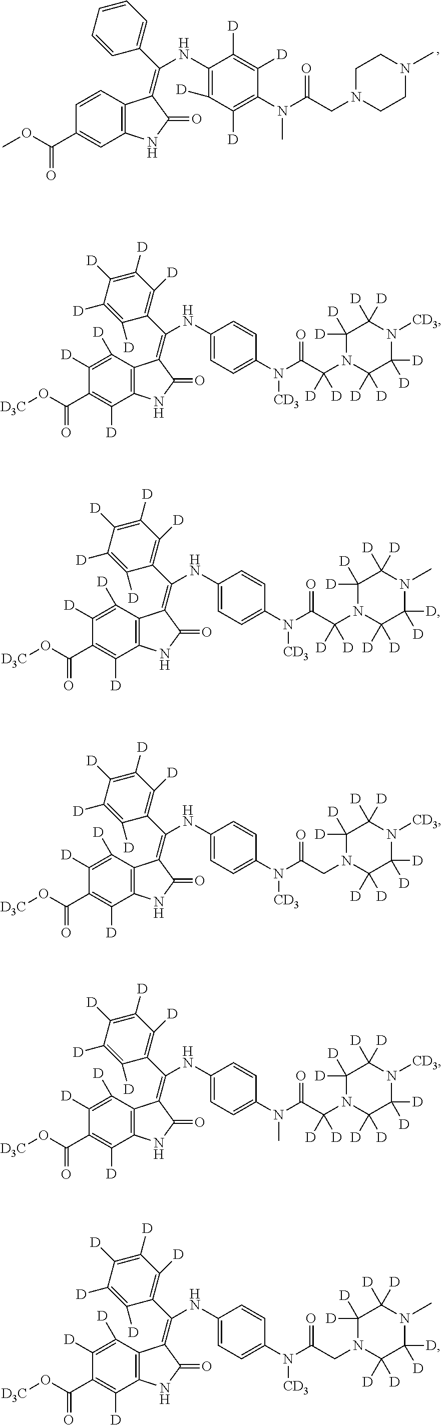

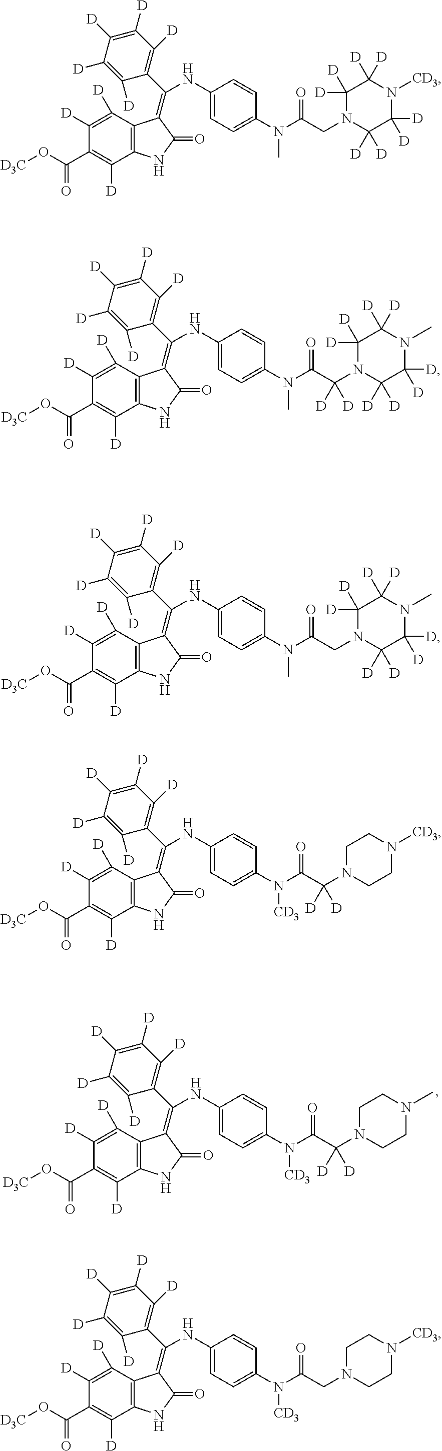

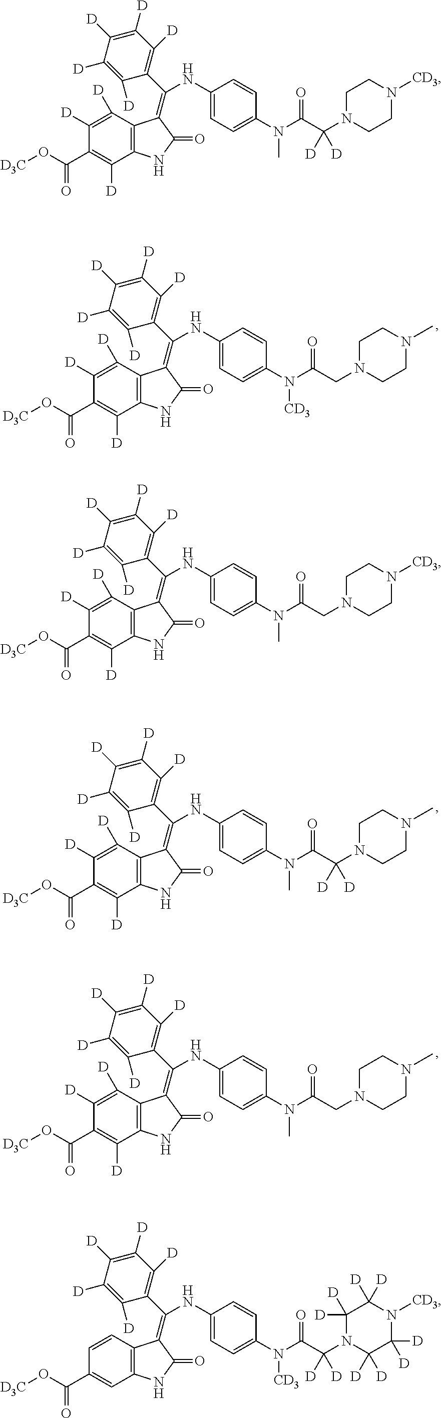

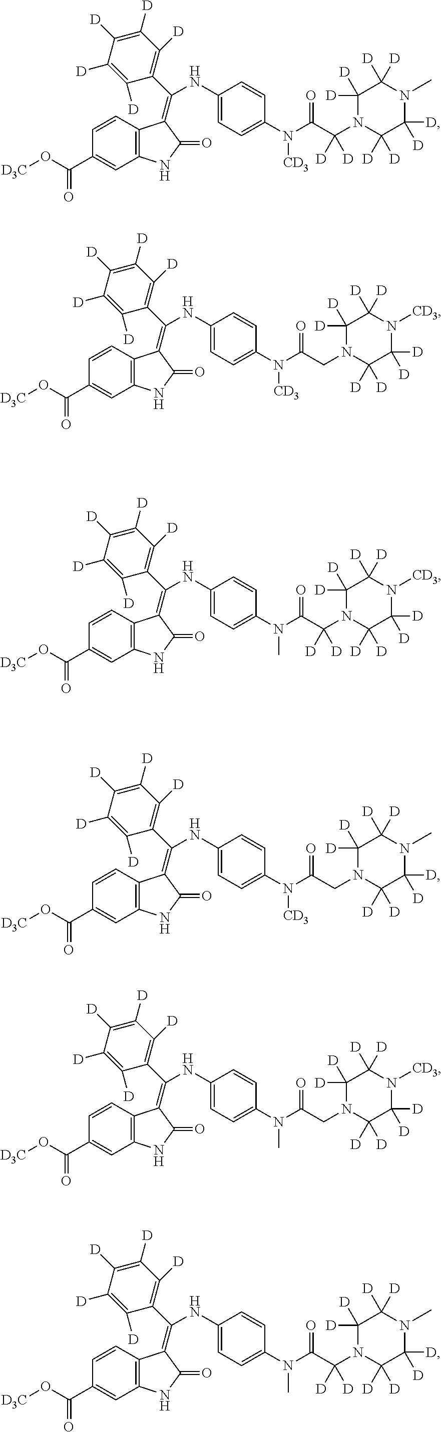

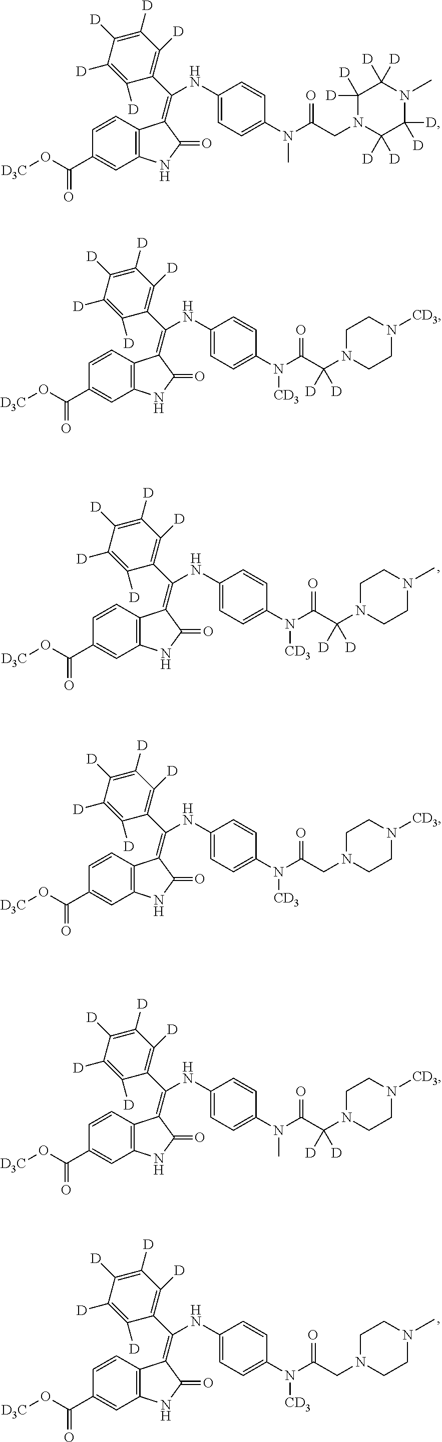

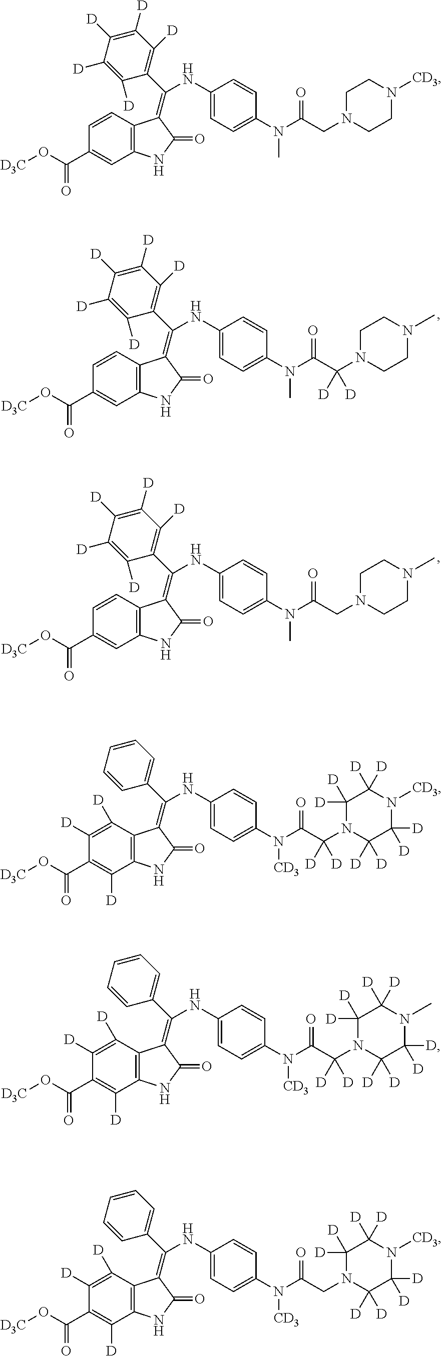

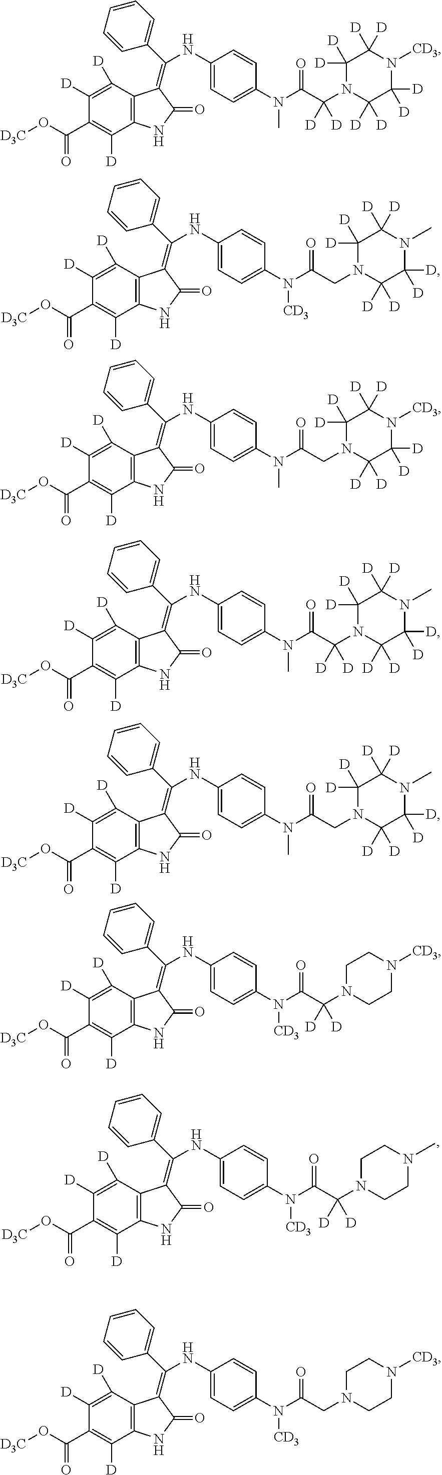

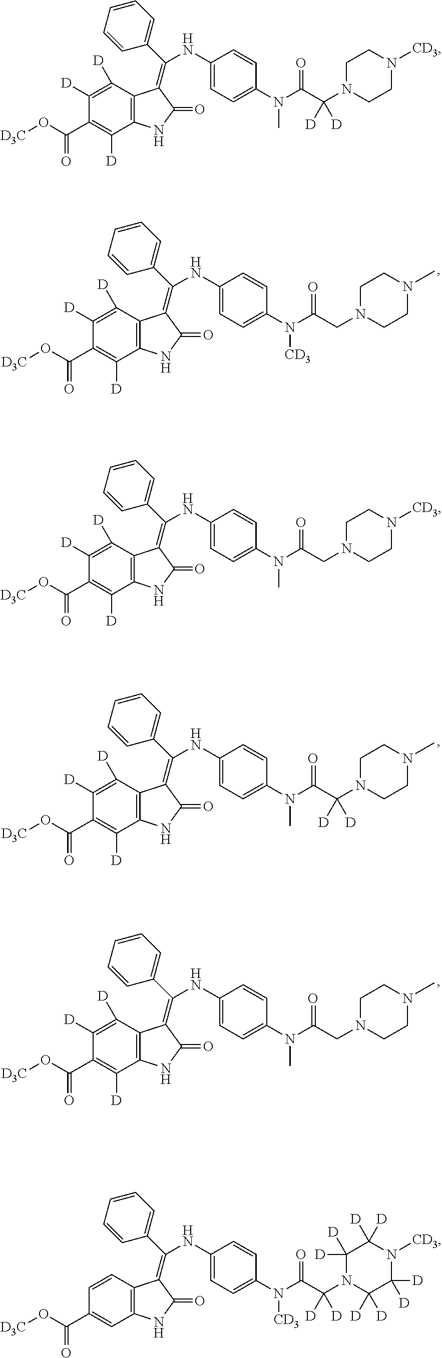

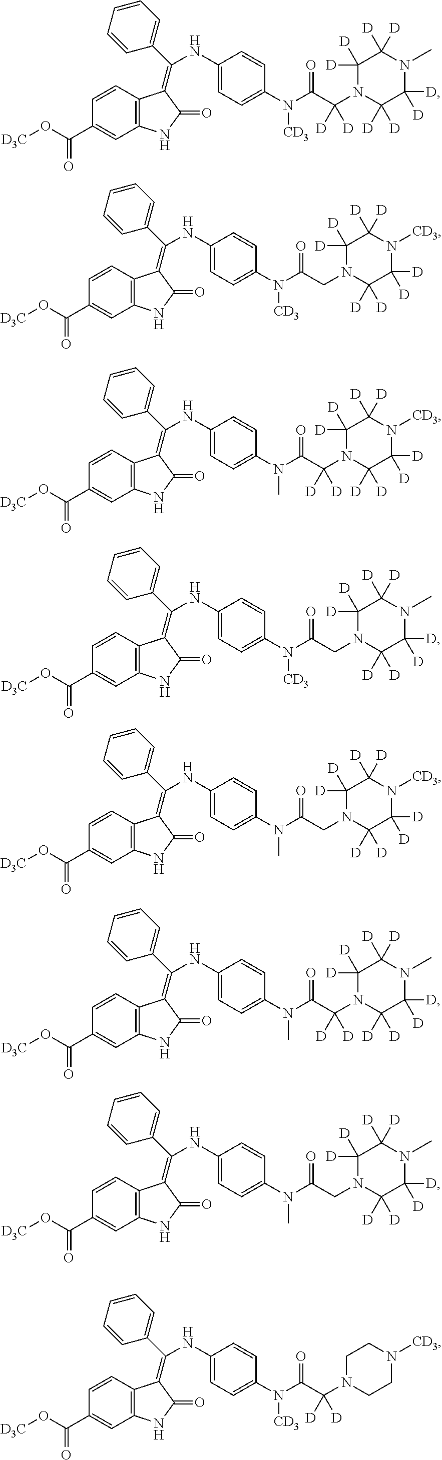

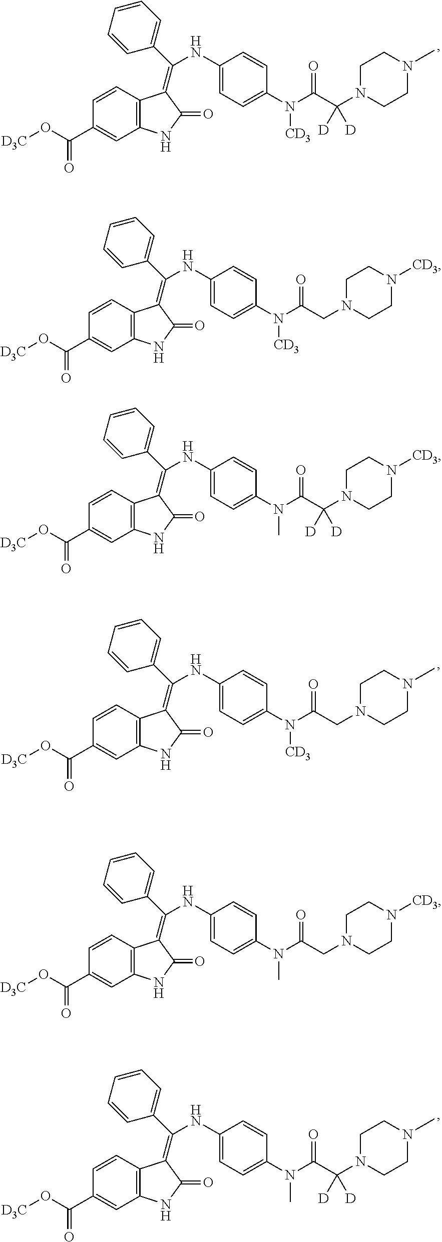

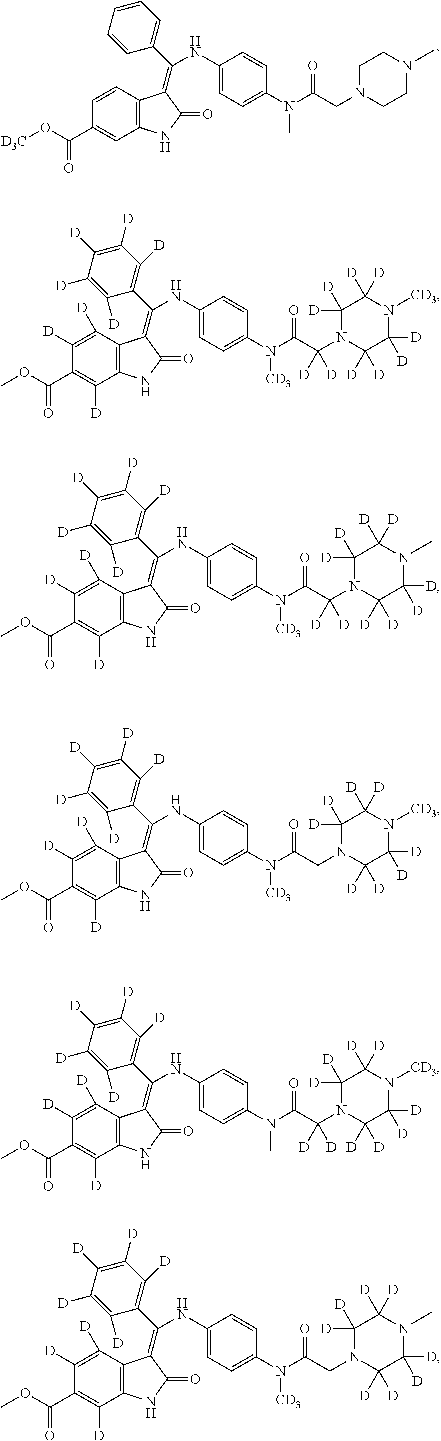

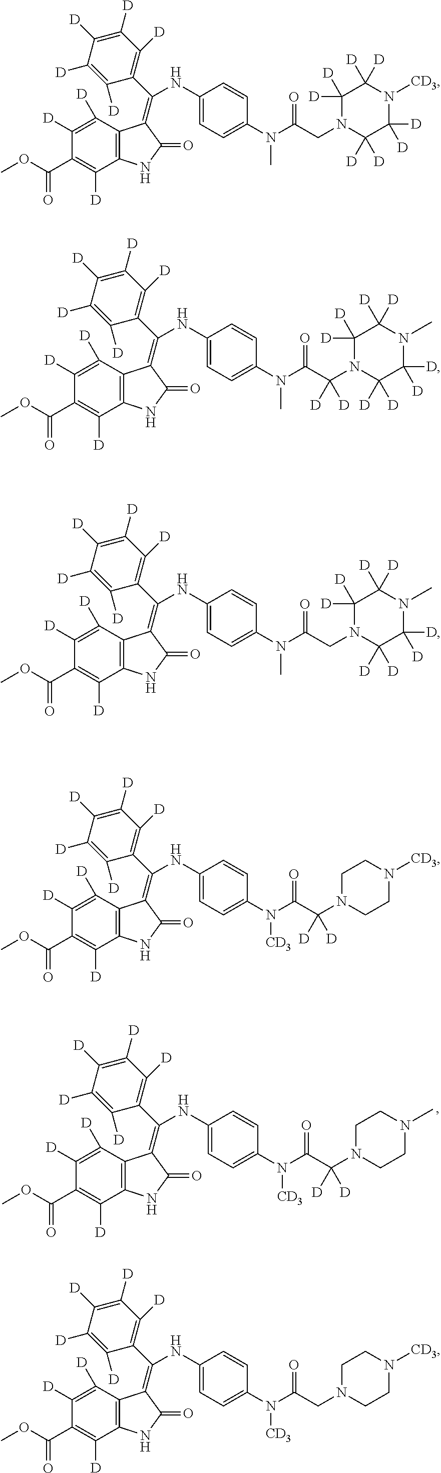

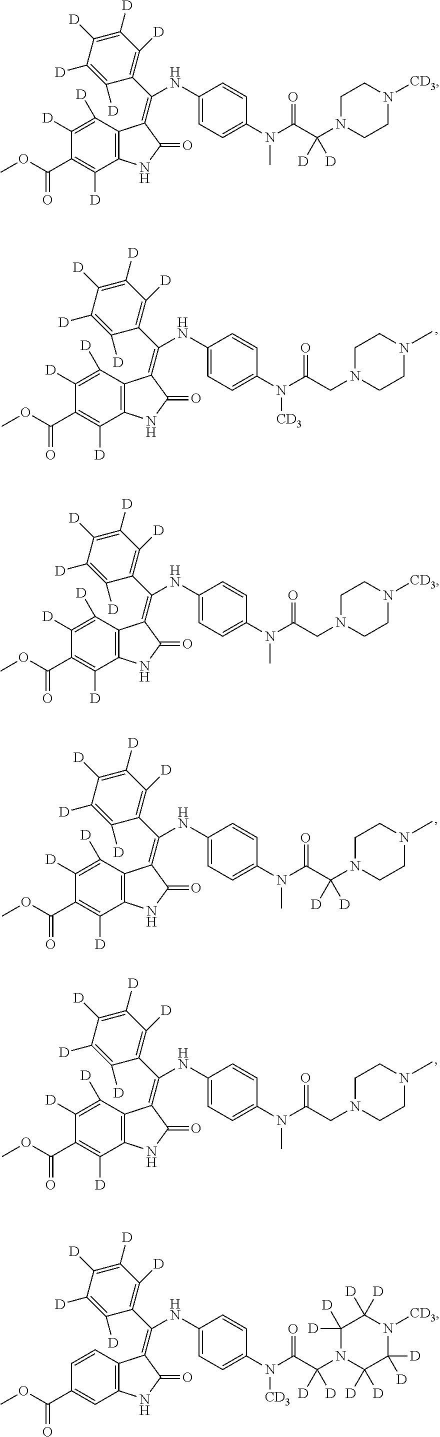

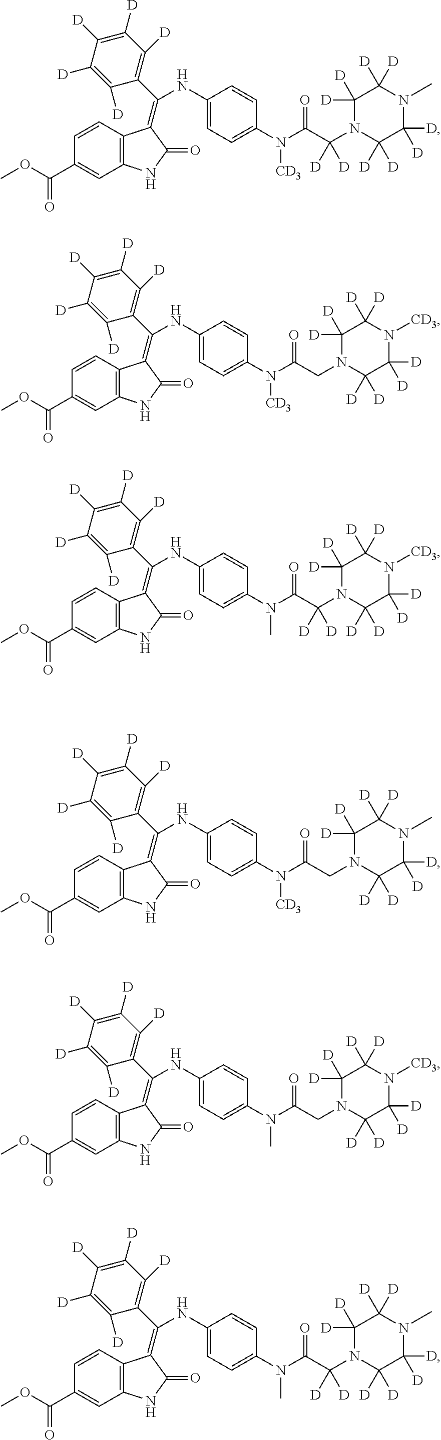

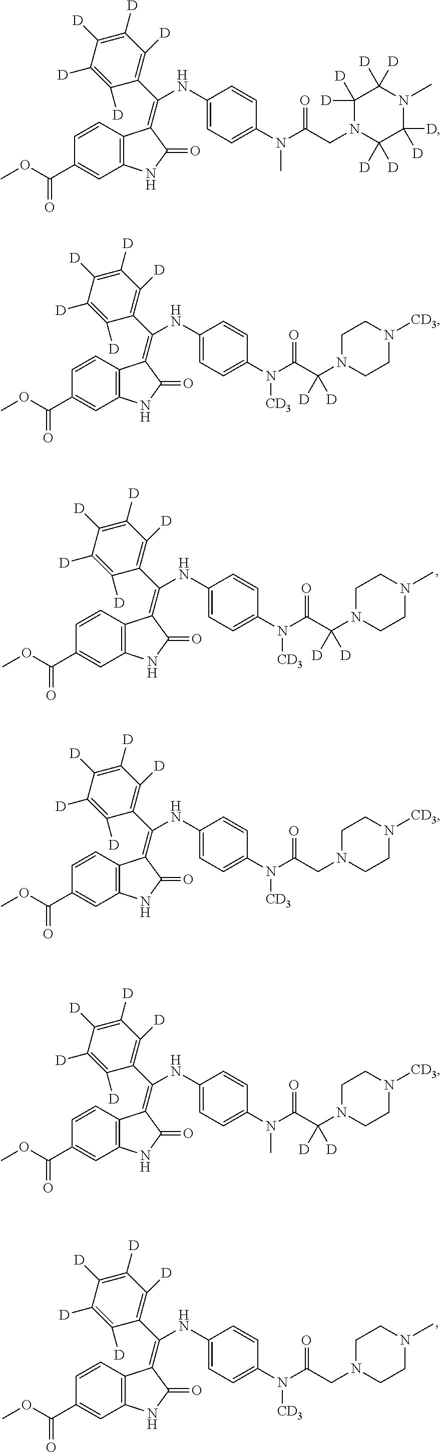

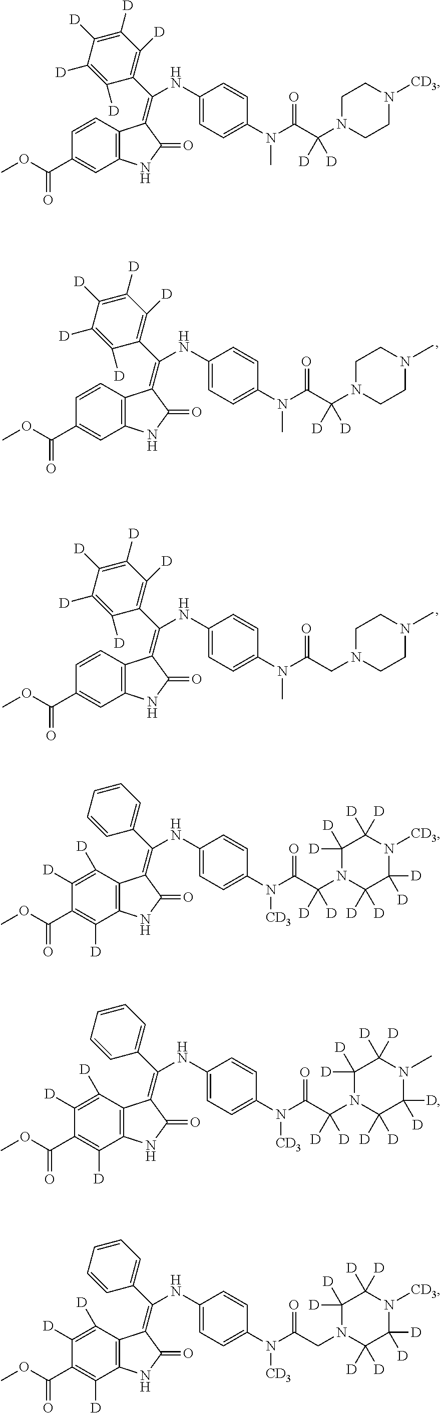

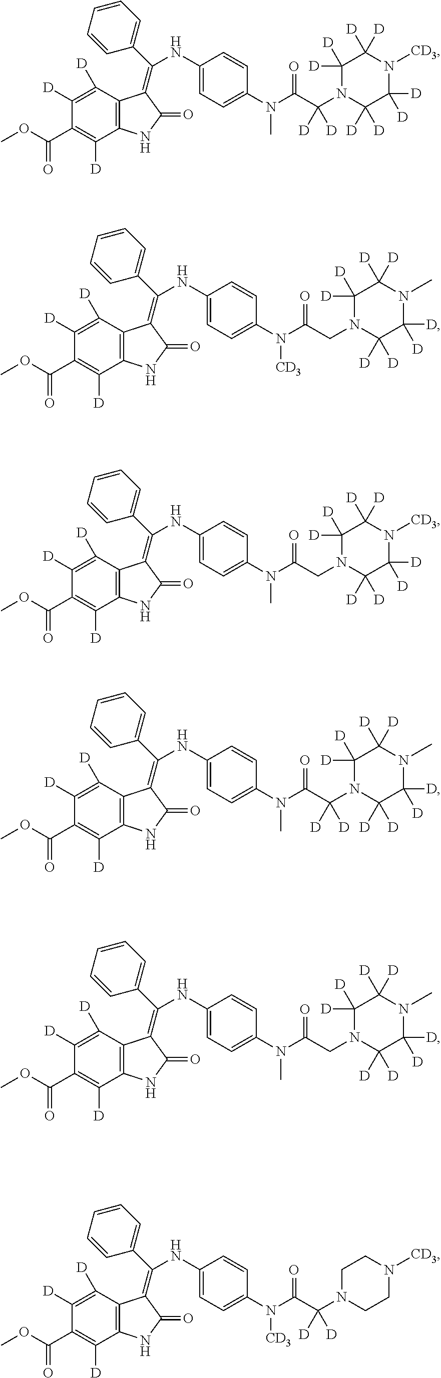

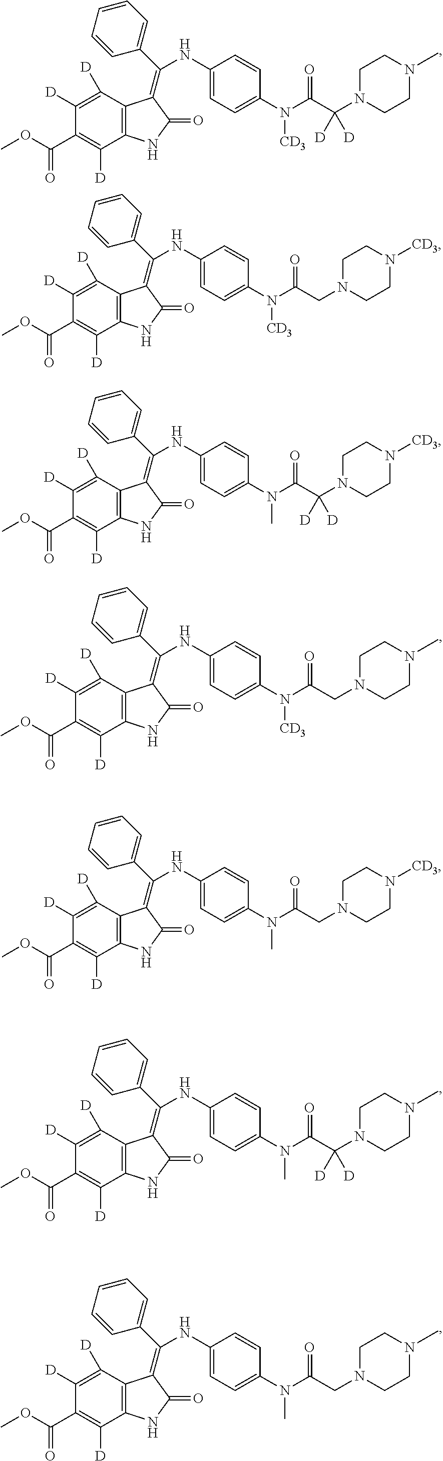

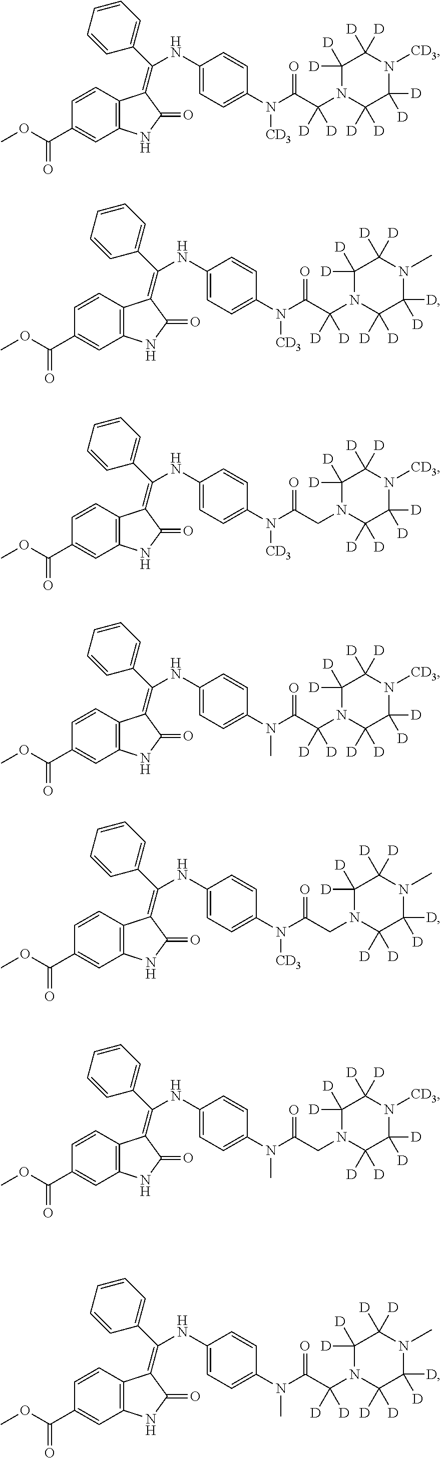

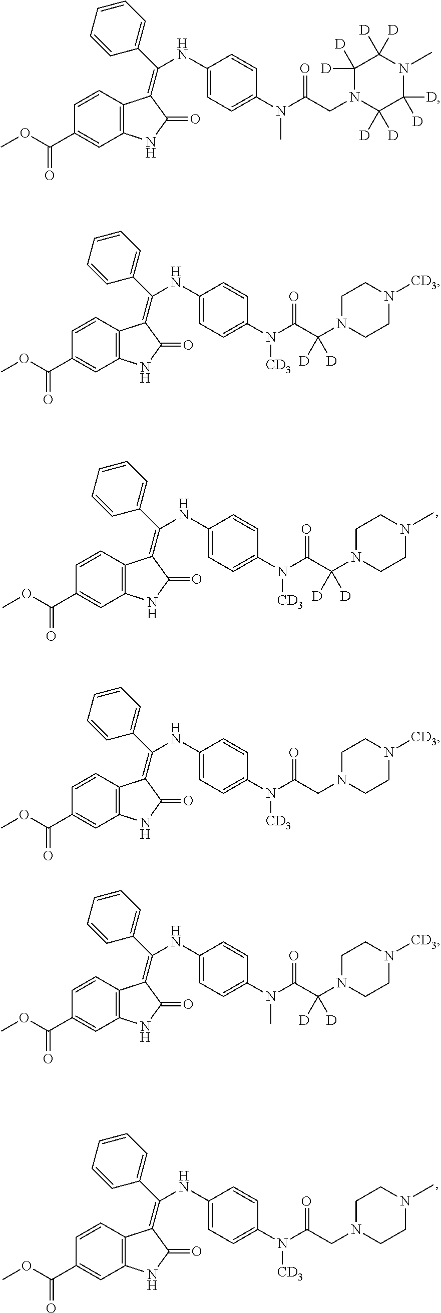

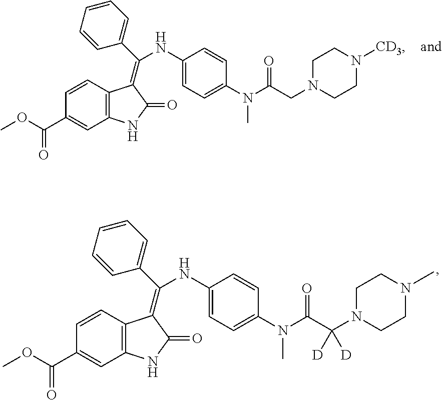

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### Biological Activity Assays

### In Vitro Liver Microsomal Stability Assay

Liver microsomal stability assays are conducted at 1 mg per mL liver microsome protein with an NADPH-generating system in 2% NaHCO₃ (2.2 mM NADPH, 25.6 mM glucose 6-phosphate, 6 units per mL glucose 6-phosphate dehydrogenase and 3.3 mM MgCl₂). Test compounds are prepared as solutions in 20% acetonitrile-water and added to the assay mixture (final assay concentration 5 microgram per mL) and incubated at 37° C. Final concentration of acetonitrile in the assay should be <1%. Aliquots (50 μL) are taken out at times 0, 15, 30, 45, and 60 min, and diluted with ice cold acetonitrile (200 μL) to stop the reactions. Samples are centrifuged at 12,000 RPM for 10 min to precipitate proteins. Supernatants are transferred to microcentrifuge tubes and stored for LC/MS/MS analysis of the degradation half-life of the test compounds.

It has been found that certain deuterium-enriched compounds disclosed herein that have been tested in this assay showed an increased degradation half-life as compared to the non-isotopically enriched drug. In certain embodiments, the increase in degradation half-life is at least 5%, at least 10%, at least 15%, or at least 20%.

### In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Monoamine Oxidase A Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

### Monooamine Oxidase B Inhibition and Oxidative Turnover

The procedure is carried out as described in Uebelhack, Pharmacopsychiatry 1998, 31(5), 187-192, which is hereby incorporated by reference in its entirety.

### In Vitro VEGFR-2 Kinase Assay

The procedure is carried out as described in Roth et al., J. Med. Chem., 2009, 52(14), 4466-4480, which is hereby incorporated by reference in its entirety.

### Non-Radioactive Kinase Assay (Ick)

The procedure is carried out as described in WO 2004017948, which is hereby incorporated by reference in its entirety.

### Bleomycin-Induced Pulmonary Fibrosis Assay

The procedure is carried out as described in WO 2006067165, which is hereby incorporated by reference in its entirety.

### Human Umbilical Endothelial Cell Proliferation Assay

The procedure is carried out as described in U.S. Pat. No. 6,762,180, which is hereby incorporated by reference in its entirety.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound having the structural formula:

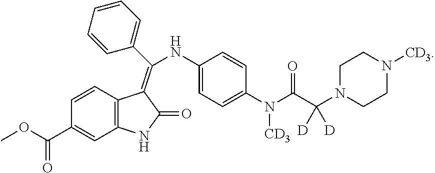 [SMILES: [2H]C([2H])([2H])N1CCN(C([2H])([2H])C(=O)N(c2ccc(N/C(=C3\C(=O)Nc4cc(C(=O)OC)ccc43)c3ccccc3)cc2)C([2H])([2H])[2H])CC1]

2. The compound as recited in claim 1 wherein each position represented as D has deuterium enrichment of no less than about 10%.

3. The compound as recited in claim 1 wherein each position represented as D has deuterium enrichment of no less than about 50%.

4. The compound as recited in claim 1 wherein each position represented as D has deuterium enrichment of no less than about 90%.

5. The compound as recited in claim 1 wherein each position represented as D has deuterium enrichment of no less than about 98%.

6. A pharmaceutical composition comprising a compound as recited in claim 1 together with a pharmaceutically acceptable carrier.

7. A compound having the structural formula:

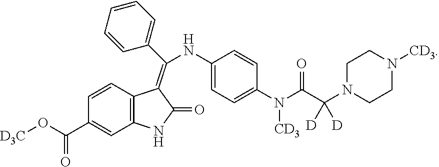 [SMILES: [2H]C([2H])([2H])OC(=O)c1ccc2c(c1)NC(=O)/C2=C(\Nc1ccc(N(C(=O)C([2H])([2H])N2CCN(C([2H])([2H])[2H])CC2)C([2H])([2H])[2H])cc1)c1ccccc1]

8. The compound as recited in claim 7 wherein each position represented as D has deuterium enrichment of no less than about 10%.

9. The compound as recited in claim 7 wherein each position represented as D has deuterium enrichment of no less than about 50%.

10. The compound as recited in claim 7 wherein each position represented as D has deuterium enrichment of no less than about 90%.

11. The compound as recited in claim 7 wherein each position represented as D has deuterium enrichment of no less than about 98%.

12. A pharmaceutical composition comprising a compound as recited in claim 7 together with a pharmaceutically acceptable carrier.
